(19) United States
(12) Patent Application Publication
(10) Pub. No.: US 2026/0294565 A1
(43) Pub. Date: Oct. 1, 2026

(54) SPATIOTEMPORAL AUTOCALIBRATION OF MULTI-SEGMENT ROBOTIC MEDICAL INSTRUMENTS

(71) Applicant: Intuitive Surgical Operations, Inc., Sunnyvale, CA (US)

(72) Inventors: Erez Posner, Rehovot (IL); Moshe Bouhnik, Holon (IL); Netanel Frank, Tel Aviv (IL); Ore Shtalrid, Shoham (IL)

(73) Assignee: Intuitive Surgical Operations, Inc., Sunnyvale, CA (US)

(21) Appl. No.: 19/631,663

(22) Filed: Mar. 27, 2026

Related U.S. Application Data
(60) Provisional application No. 63/779,721, filed on Mar. 28, 2025.

(51) Int. Cl. A61B 34/32; A61B 17/00; A61B 34/30; A61B 90/00

(52) U.S. Cl. CPC A61B 34/32; A61B 17/00; A61B 90/37; A61B 2017/00725; A61B 2034/301; A61B 2034/306

(57) ABSTRACT

The technical solutions are directed to a spatial and temporal autocalibration of a multi-segment instrument system used in medical procedures. A system can include a processor coupled with memory to receive, from a shape sensor system, pose information related to a tip of a segment of a body of an instrument used to perform a medical procedure. The processor can receive, from a vision system, a video of the medical procedure and detect an out-of-calibration error between one or more frames of the video stream and the pose information. The processor can apply, responsive to detection of the out-of-calibration error during the medical procedure, a compensation matrix to one of the pose information or the video stream to align the pose information with the video stream. The processor can execute an action of the medical procedure using the pose information aligned with the video stream based on the compensation matrix.

### CROSS-REFERENCES TO RELATED APPLICATIONS

This application claims benefit and priority under 35 U.S.C. § 119 to U.S. Provisional Patent Application No. 63/779,721, filed Mar. 28, 2025, which is hereby incorporated by reference herein in its entirety.

### BACKGROUND

Medical procedures can be performed in an operating room using various medical instruments, systems and tools. As the amount and variety of equipment in the operating room increases, or medical procedures become increasingly complex, it can be challenging to perform such procedures efficiently, reliably, or without incident.

### SUMMARY

The technical solutions described herein are directed to a spatial and temporal autocalibration of instrument systems used in robotic medical procedures. Instrument systems, such as flexible multi-segment endoscopic devices, can be used in medical procedures for moving through a narrow path within a patient's body to perform various tasks, such as capturing of an image, acquiring a tissue sample, or administering a treatment. In such configurations, image capture devices, such as video cameras, can be used together with the multi-segment instruments to perform various medical tasks. In order to perform such tasks accurately and reliably, it is important that these instrument systems and the image capture devices remain calibrated throughout the procedure both spatially and temporally. Falling out of calibration during the procedure, such as due to an impact or temperature changes, can impact decisions made by the surgeon, potentially affecting the performance during the procedure.

The technical solutions overcome these challenges by providing a spatiotemporal autocalibration of the multi-segment instruments that can be performed during the procedure itself. The technical solutions can monitor the data from the instrument sensors and the video cameras to identify a location or a pose of a tip of a multi-segment instrument using both the sensor and the video data. Upon detecting a mismatch between the sensor and the video data with respect to the location or pose of the instrument tip, the technical solutions can determine that the system is out of calibration. In order to recalibrate the system, the technical solutions can generate and apply a compensation matrix for either the sensor or the video data to realign the sensor data with the video data and recalibrate the system. The solutions can then continue execution medical procedure actions using the compensation matrix, thereby maintaining the calibration through the ongoing medical procedure.

An aspect of the technical solutions is directed to a system. The system can include one or more processors, coupled with memory. The one or more processors can be configured (e.g., via instructions or data stored in the memory) to receive, from a shape sensor system, pose information related to a tip of one or more segments along a flexible body of an instrument system used to perform a medical procedure. The one or more processors can be configured to receive, from a vision system, a video stream related to the medical procedure. The one or more processors can be configured to detect, during the medical procedure, an out-of-calibration error between one or more frames of the video stream and the pose information. The one or more processors can be configured to apply, responsive to detection of the out-of-calibration error during the medical procedure, a compensation matrix to at least one of the pose information or the video stream to align the pose information with the video stream. The one or more processors can be configured to execute an action related to the medical procedure using the pose information aligned with the video stream based on the compensation matrix.

The shape sensor and the vision system can be calibrated prior to performance of the medical procedure using a factory-calibrated transform matrix. The factory-calibrated transform matrix can be different from the compensation matrix, which can be used or generated during the medical procedure responsive to detection of the out-of-calibration error. The one or more processors can be configured to temporally synchronize frames of the pose information and frames of the video stream by applying one or more offsets to one or more stamps associated with at least one of the frames of the pose information or the frames of the video stream. The one or more processors can be configured to apply the compensation matrix to align timestamps associated with frames of the pose information and frames of the video stream.

The one or more processors can be configured to receive, from one or more sensors, kinematics information during the medical procedure. The one or more processors can be configured to detect an out-of-calibration error between the kinematics information and at least one of the pose information or the video stream. The one or more processors can be configured to apply a second compensation matrix to kinematics data to align the kinematics data with frames of the at least one of the pose information or the video stream. The one or more processors can be configured to apply the second compensation matrix to sensor data with the frames of the at least one of the pose information or the video stream. The one or more processors can be configured to apply the second compensation matrix to perform hand eye calibration of the frames of the kinematics information with the frames of the at least one of the pose information or the video stream.

The one or more processors can be configured to activate, subsequent to application of the compensation matrix, a digital ruler to measure a dimension of an anatomical structure related to the medical procedure. The one or more processors can be configured to provide, for display via a display device, an indication of a measurement of the dimension made using the digital ruler.

The one or more processors can be configured to register, based on the calibrated pose information and the video stream using the compensation matrix, a virtual object related to the medical procedure. The one or more processors can be configured to display, based on the calibrated pose information and the video stream using the compensation matrix, a virtual annotation with the video stream of the medical procedure.

The one or more processors can be configured to detect the out-of-calibration error based on camera intrinsic parameters. The one or more processors can be configured to identify a type of task performed during the medical procedure and select a calibration threshold based on the type of task. The one or more processors can be configured to determine the out-of-calibration error based on the calibration threshold selected for the type of task.

The one or more processors can be configured to select a calibration technique to apply based on the type of task. The one or more processors can be configured to determine the compensation matrix to apply based on the selected calibration technique. The calibration technique can include a rigid transform per frame. The calibration technique can include an interactive parametric model configured based on a state of a tool coupled with the instrument system.

An aspect of the technical solutions is directed to a method. The method can include one or more processors coupled with memory receiving, from a shape sensor system, pose information related to a tip of one or more segments along a flexible body of an instrument system used to perform a medical procedure. The method can include the one or more processors receiving, from a camera, a video stream related to the medical procedure. The method can include the one or more processors detecting, during the medical procedure, an out-of-calibration error between one or more frames of the video stream and the pose information. The method can include the one or more processors calibrating, responsive to detection of the out-of-calibration error during the medical procedure, the pose information with the video stream using a compensation matrix. The method can include the one or more processors providing, for display via a display device, a location of the tip aligned based on application of the compensation matrix.

The method can include the one or more processors temporally synchronizing frames of the pose information and frames of the video stream by applying one or more offsets to one or more timestamps associated with at least one of the frames of the pose information or the frames of the video stream. The method can include the one or more processors receiving, from one or more sensors, kinematics information during the medical procedure. The method can include detecting, by the one or more processors, an out-of-calibration error between the kinematics information and at least one of the pose information or the video stream. The method can include applying, by the one or more processors, a second compensation matrix to the kinematics information to align frames of the kinematics information with frames of the at least one of the pose information or the video stream.

The method can include activating, by the one or more processors, subsequent to application of the compensation matrix, a digital ruler to measure a dimension of an anatomical structure related to the medical procedure. The method can include providing, for display via the display device, an indication of a measurement of the dimension made using the digital ruler. The method can include registering, by the one or more processors, based on the calibrated pose information and the video stream using the compensation matrix, a virtual object related to the medical procedure.

An aspect of the technical solutions is directed to a non-transitory computer-readable medium storing processor-executable instructions. The instructions, when executed by one or more processors, can cause the one or more processors to receive, from a shape sensor system, position and orientation information related to a tip of one or more segments along a flexible body of an instrument system used to perform a medical procedure. The instructions, when executed by one or more processors, can cause the one or more processors to receive, from a sensor, a video stream related to the medical procedure. The instructions, when executed by one or more processors, can cause the one or more processors to detect, during the medical procedure, an out-of-calibration error between one or more frames of the video stream and the position and orientation information. The instructions, when executed by one or more processors, can cause the one or more processors to align, responsive to detection of the out-of-calibration error during the medical procedure, the position and orientation information with the video stream using a compensation matrix. The instructions, when executed by one or more processors, can cause the one or more processors to perform an action related to the medical procedure using the aligned position and orientation information with the video stream.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing. In the drawings:

FIG. 1 depicts an example system for providing spatial and temporal automated calibration of multi-segment instrument systems in robotic medical procedures.

FIG. 2 illustrates an example of a surgical system, in accordance with some aspects of the technical solutions.

FIG. 3 illustrates an example block diagram of an example computer system is shown, in accordance with some aspects of the technical solutions.

FIG. 4. illustrates an example of a flow diagram of a method for providing a factory calibration of the instrument system.

FIG. 5 illustrates an example of plots of temporally non-calibrated and temporally calibrated instrument systems.

FIG. 6 illustrates an example of a configuration example for a factory calibration.

FIG. 7 illustrates an example of a configuration example for performing robot to camera factory calibration.

FIG. 8 illustrates an example of a configuration example for performing final joint optimization for a factory calibration.

FIG. 9 illustrates an example flow diagram of a method for providing an instrument recalibration during an ongoing medical procedure.

FIG. 10 illustrates an example of a recalibrated instrument illustrated from different viewpoints.

FIG. 11 illustrates an example of a flow diagram of a recalibration of an instrument during a procedure.

FIG. 12 illustrates an example of an instrument system that can be calibrated using the techniques described herein.

FIG. 13 illustrates an example flow diagram of a method for providing a spatial or temporal autocalibration of a multi-segment instrument system for a medical procedure.

### DETAILED DESCRIPTION

Following below are more detailed descriptions of various concepts related to, and implementations of, systems, methods, apparatuses for spatial and temporal autocalibration of instrument systems used in robotic medical procedures. The various concepts introduced above and discussed in greater detail below can be implemented in any of numerous ways.

Although the present disclosure is discussed in the context of a surgical procedure, in various aspects, the technical solutions of this disclosure can be applicable to other medical or non-medical applications, treatments, sessions, environments or activities, in which calibration of multi-segment instruments during performance of a procedure is sought. For instance, technical solutions can be applied in any environment, application or industry in which activities, operations, processes or acts by robots or robotic tools involve or utilize recalibration of the instrument systems user for performance of an ongoing procedure.

The technical solutions address the challenges of maintaining calibration between various sensors and components of instrument systems that can be used for performing robotic medical procedures, such as robotic surgeries. In the context of medical procedures, it can be important ensure that the instruments and imaging devices used for medical procedure task implementation remain accurately calibrated to provide precise and reliable data. This becomes even more pronounced in multi-segment instruments, such as flexible endoscopic devices, which can be used to navigate through narrow pathways within a patient's body. The calibration of these instruments with the vision system used for their guidance and operation allows for accurate tracking and execution of medical tasks during the procedure.

The technical challenges arise when there is a mismatch between the data from the shape sensor system and the video stream from the vision system. This out-of-calibration error can occur due to various factors, such as movement of the instrument, changes in the environment, temperature, stress or pressure, as well as due to inherent inaccuracies in the sensors. These discrepancies can impact decisions made by the surgeon potentially affecting the performance of the medical procedure. As a result, there can be instances during the medical procedure when it is important to detect and automatically such calibration errors between the sensor and video data in real-time and during the ongoing procedure.

The technical solutions can overcome such challenges by providing devices, apparatuses systems and methods for spatial-temporal automated autocalibration of instrument systems. The technical solutions can provide processors with memories to receive sensor information (e.g., position information, orientation information, or pose information) from a sensor system and video data from a video stream of a vision system capturing the medical procedure. The sensor system can include a shape sensor system or any set of one or more sensors that are configured to provide signaling on pose, orientation, movement or position of a medical system or a device. The solutions can utilize such sensor and video data to detect an out-of-calibration error between the video stream and the pose information. Upon detecting that the instrument system is to be recalibrated, the solutions can generate and apply one or more compensation matrices to align the sensor data (e.g., the pose information) with the data in the video stream. This realignment can adjust the instrument's position and orientation to be accurately represented in the video stream, allowing for precise execution of medical tasks in the medical procedure. The solutions can continue to monitor the sensor and video data, and continue to adjust the calibration throughout the procedure, as needed, maintaining the accuracy and reliability of the instrumentation during the procedure.

Thus, the data processing system described herein can improve the technical functioning of an instrument system and associated vision system during an ongoing procedure by integrating or executing, for example, rigid transforms, offsets, or matrix-based compensation. The data processing system can use the pose information from the shape sensor system and the video stream (including frames and associated timestamps) to detect an out-of-calibration error during the procedure and, responsive to that detection, and apply a compensation matrix to at least one of the pose information or the video stream to realign the pose information with the video stream. This real-time recalibration allows the system to continue operating with calibrated spatial and temporal alignment despite changes that arise during the procedure (e.g., impacts, environmental changes, temperature, stress, or sensor drift), thereby maintaining the accuracy and reliability of the calibrated instrument representation used by the robotic medical system and its associated visualization tools.

The data processing system described herein can facilitate or improve the execution of downstream operations, including execution of actions with the instrument system using the pose information aligned with the video stream based on the compensation matrix, by providing calibrated alignment and calibrated visualization functions, such as a digital ruler measurement and registration or display of virtual objects or virtual annotations with the video stream. As such, by using a compensation matrix and synchronization operations (e.g., applying one or more offsets to timestamps to temporally align frames of pose information and frames of the video stream), the data processing system can provide a real-time calibration pipeline that produces an improved and continually corrected alignment between pose derived from sensors and camera imagery obtained during the procedure. Accordingly, to address technical challenges arising from calibrating instrument systems with multiple segments, the data processing system described herein can provide specific technical solutions including automatically detecting and correcting out-of-calibration errors, and using the corrected alignment for instrument control and calibrated visualization during performance of the procedure, for example.

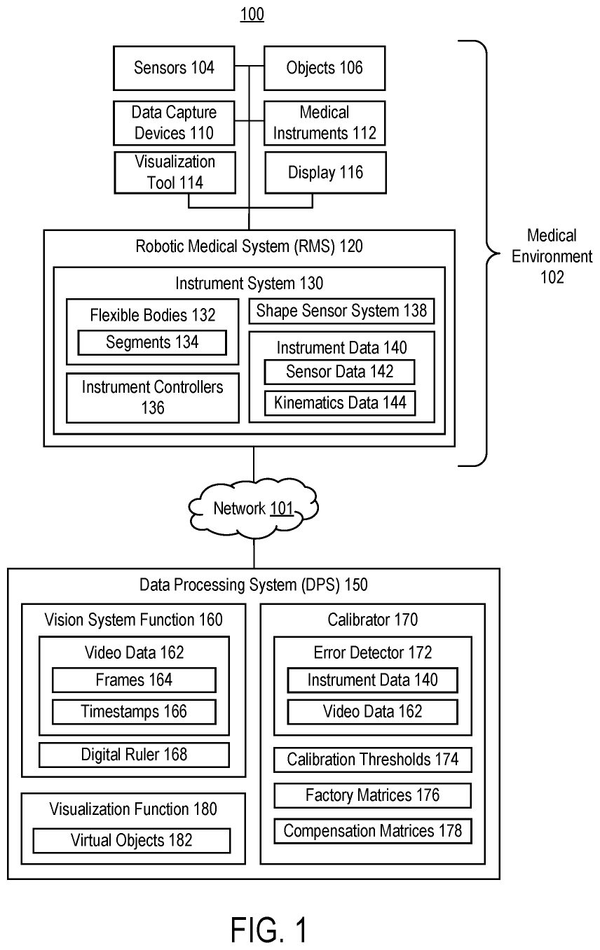

FIG. 1 depicts an example system 100 for providing spatial and temporal automated calibration of multi-segment instrument systems in robotic medical procedures. The example system 100 can include a medical environment 102 in which a medical procedure can take place. The medical environment 102 can include one or more of: sensors 104 for sensing or detection of various tools or persons, objects 106 (e.g., medical beds or furniture, stands or supports), data capture devices 110 (e.g., cameras), medical instruments 112 for performing the procedure, visualization tools 114 (e.g., multi-data simulators or visualizers), displays 116 for displaying data, or robotic medical systems 120. The robotic medical system (RMS) 120 can be communicatively coupled, such as via a network 101, with a data processing system (DPS) 150.

The robotic medical system 120 can include or be coupled with one or more instrument systems 130, such as a multi-segment, flexible-body endoscopic instrument. The instrument system 130 can include one or more of flexible bodies 132 that can be composed of one or more segments 134. The instrument system 130 can include one or more instrument controllers 136 for controlling the medical instruments 112 that can be attached to and manipulated by the instrument system, such as to execute actions related to the medical procedure being performed according to the recalibration. The instrument system 130 can include one or more shape sensor systems 138 for capturing or processing instrument data 140 on the movement of the segments 134, including for example pose information on a tip of a particular segment 134. The instrument data 140 can include, for example, sensor data 142 and kinematics data 144.

The data processing system 150 can include one or more of vision system functions 160, calibrators 170 and visualization functions 180. The vision system function 160 can receive, gather or process image data, including video data 162 of the medical procedure, which can include various video frames 164 and their associated timestamps 166. The vision system function 160 can include one or more digital rulers 168 that can be provided for measuring dimensions of anatomical structures in the medical procedure and displayed via a display 116. The calibrator 170 can include one or more error detectors 172 for detecting out-of-calibration errors based on instrument data 140 and video data 162 based on various calibration thresholds 174. The calibrator 170 can include or generate one or more factory matrices 176 and compensation matrices 178 (e.g., for recalibration in response to out-of-calibration errors) to align the instrument data 140 (e.g., sensor data indicating the pose information) with the video stream data. Once generated, the compensation matrices 178 can be applied by the instrument controllers 136 for instrument system 130 operation, such as to execute actions related to the ongoing medical procedure using the pose information aligned with the video stream based on a compensation matrix 178. The compensation matrices 178 can also be utilized for displaying, based on the recalibration, virtual objects 182 (e.g., virtual features or annotations) generated by a visualization function 180.

Medical environment 102 can include any area, region or setting in which a medical procedure can take place. Medical environment 102 can include, for example, operating rooms, clinics, or specialized medical facilities which can be equipped with various equipment, tools, devices, and systems for performing the medical procedures. For instance, medical environment102 can include a surgical room that includes various combination of sensors 104 (, objects 106, data capture devices 110, medical instruments 112, visualization tools 114 and displays 116. Within this environment, the system can ensure that all components are properly calibrated to maintain accuracy during procedures. The medical environment 102 can also support the integration of advanced technologies, such as robotic systems and data processing units, to enhance the precision and efficiency of medical procedures. For example, the medical environment 102 can facilitate the use of real-time data from sensors and cameras to monitor and adjust the calibration of medical instruments during a procedure.

The medical environment 102 can include any arrangement of sensors 104, objects 106, data capture devices 110, medical instruments 112, visualization tools 114 and displays 116 utilized with the robotic medical systems 120 to perform a medical procedure. The objects 106 can include any type of objects or articles, such as medical operating tables, shelves, holders, various medical instruments separate from those used by the robotic medical systems 120, surgical lights, medical equipment carts, imaging equipment or other systems or tools for carrying fluids or patient monitoring equipment. The data capture devices 110 (e.g., optical devices, such as image or video cameras, as well as microphones, radio frequency identification (RFID) readers, data loggers, smartphone or tablet devices or depth sensors) can be used for logging or capturing any data streams. The data streams can include any sequence or stream of data, including sequence or stream of any sensor data 142 (e.g., data from sound sensors, video cameras or other sensors), events data (e.g., data on logs of events or occurrences involving a robotic medical systems 120) and kinematics data 144 (e.g., movements of medical instruments 112). For example, the instrument data 140 can include a stream of events data (e.g., event stream) which can provide event information that can be timestamped and used with sensor and kinematics data for recalibrating instrument systems. The event stream data can include or identify actions or occurrences involving the instrument system according to timestamps.

Sensors 104 can include any devices used for sensing or detecting various tools, persons, or conditions within the medical environment 102. These sensors can capture data related to the position, movement, and status of medical instruments and other objects. For example, sensors 104 can include shape sensors that provide pose information about the tip of a multi-segment instrument. The data collected by sensors 104 can be used to detect out-of-calibration errors and trigger recalibration processes. These sensors can be integrated with other components of the system to ensure accurate and reliable data collection during medical procedures. For example, sensors 104 can work in conjunction with vision systems to provide comprehensive monitoring and calibration of medical instruments.

Objects 106 can include any items within the medical environment 102 that can be used to facilitate a medical procedure, such as medical beds, furniture, stands, or supports. Objects 106 can include medical shelves, drawers, holders of medical supplies or equipment, patient's bed, medical carts, any of which can be positioned to facilitate the performance of medical procedures. For example, objects 106 can include infusion poles that hold IV bags and support tubing for administering fluids to patients. The placement and configuration of objects 106 can be adjusted or optimized to ensure easy access and efficient workflow during procedures. Objects 106 can be equipped with sensors 104 or data capture devices 110 to monitor their position and status, contributing to the overall calibration and accuracy of the system.

Data capture devices 110 can include any devices used to capture data related to the medical procedure. Data capture devices 110 can include cameras, microphones, and other recording equipment. For example, data capture devices 110 can include high-resolution cameras that provide a video stream of the medical procedure. The data captured by the data capture devices 110 can include images or video frames 164 that can be timed using timestamps 166 and can be used to monitor and analyze the procedure in real-time. Data capture devices 110 can be integrated with other components of the system 100 to ensure accurate and comprehensive data collection. For example, data capture devices 110 can provide data for or of, or work in conjunction with, sensors 104 and vision systems (e.g., vision system functions 160) to detect out-of-calibration errors and trigger recalibration processes.

Data capture devices 110 can include any of a variety of sensors, cameras, video imaging devices, infrared imaging devices, visible light imaging devices, intensity imaging devices (e.g., black, color, grayscale imaging devices, etc.), depth imaging devices (e.g., stereoscopic imaging devices, time-of-flight imaging devices, etc.), medical imaging devices such as endoscopic imaging devices, ultrasound imaging devices, etc., non-visible light imaging devices, any combination or sub-combination of the above mentioned imaging devices, or any other type of imaging devices that can be suitable for the purposes described herein. Data capture devices 110 can include cameras that a surgeon can use to perform a surgery and observe manipulation components within a purview of field of view suitable for the given task performance.

Data capture devices 110 can capture, detect, or acquire sensor data, such as videos or images, including for example, still images, video images, vector images, bitmap images, other types of images, or combinations thereof. The data capture devices 110 can capture the images at any suitable predetermined capture rate or frequency. Settings, such as zoom settings or resolution, of each of the data capture devices 110 can vary as desired to capture suitable images from any viewpoint. For instance, data capture devices 110 can have fixed viewpoints, locations, positions, or orientations. The data capture devices 110 can be portable, or otherwise configured to change orientation or telescope in various directions. The data capture devices 110 can be part of a multi-sensor architecture including multiple sensors, with each sensor being configured to detect, measure, or otherwise capture a particular parameter (e.g., sound, images, or pressure).

Data capture devices 110 can include any type and form of a sensor 104 that can be configured to measure and provide sensor data 142, including a positioning sensor, a biometric sensor, a velocity sensor, an acceleration sensor, a vibration sensor, a motion sensor, a pressure sensor, a light sensor, a distance sensor, a current sensor, a focus sensor, a temperature sensor, a haptic or tactile sensor or any other type and form of sensor used for providing data on medical tools 112, or data capture devices (e.g., optical devices). Sensor 104 can include a depth sensor configured to determine a distance between the sensor and an object (e.g., distance to a medical instrument 112 or a patient's anatomy). For example, a data capture device 110 can include a location sensor, a distance sensor or a positioning sensor providing coordinate locations of a medical tool 112 or a data capture device 110. Data capture device 110 can include a sensor providing information or data on a location, position or spatial orientation of an object (e.g., medical tool 112 or a lens of data capture device 110) with respect to a reference point. The reference point can include any fixed, defined location used as the starting point for measuring distances and positions in a specific direction, serving as the origin from which all other points or locations can be determined.

Medical instruments 112 can include any tools or devices used to perform medical procedures. Medical instruments 112 can be attached to and manipulated by the instrument system 130. For example, medical instruments 112 can include various surgical tools (e.g., clippers, scissors, scalpels or needles), devices or instrument systems 130 (e.g., endoscopes, laparoscopes, bronchoscopes, cystoscopes or laparoscopic trocars), and other specialized devices. Medical instruments 112 be handled or manipulated by arms, holders or handlers of the robotic medical system 120 and can be calibrated and aligned with the video data 162 and instrument data 140. Such calibrations can be maintained throughout the procedure using autocalibrations to ensure precise and reliable performance throughout the procedure. For example, medical instruments 112 can be controlled by instrument controllers to execute actions related to the medical procedure based on real-time data from sensors and cameras.

Medical instruments or tools 112 can be imaged by, associated with or include an image capture device and can be handled or maneuvered using robotic manipulator arms 235 of the robotic medical systems 120. For instance, a medical instrument 112 can be a tool for making incisions, a tool for suturing a wound, an endoscope for visualizing organs or tissues, an imaging device, a needle and a thread for stitching a wound, a surgical scalpel, forceps, scissors, retractors, graspers, or any other tool or instrument to be used during a surgery. Medical instruments 112 can include hemostats, trocars, surgical drills, suction devices or any instruments for use during a surgery. The medical instrument 112 can include other or additional types of therapeutic or diagnostic medical imaging implements. The medical instrument 112 can be configured to be installed in, coupled with, or manipulated by a robotic medical systems 120, such as by manipulator arms 235 or other components for holding, using and manipulating the medical instruments 112 during procedure.

Visualization tool 114 can include any tools or software used to visualize data related to the medical procedure, including any functionalities for facilitating or providing simulations, visualizations or virtual annotations. For example, visualization tool 114 can include multi-data simulators that combine data from sensors 104, medical instruments 112, data capture devices 110 (e.g., cameras), and other sources to create a comprehensive view of the procedure. The robotic medical system 120 can use such visualizations from the visualization tool 114 to monitor and adjust the calibration of medical instruments 112 being utilized by the instrument system 130. For example, visualization tool 114 can process data from sensors 104, shape sensor system 138, data capture devices 110, various instrument data 140 or video data 162 to generate and display virtual objects 182 (e.g., annotations) based on the calibrated pose information and video stream. These visualizations can be used to display or improve the precision and efficiency of the procedure by providing clear and accurate information to the medical team. The visualization tools 114 can gather the captured data streams (e.g., 140 or 162) and process the data for display to the user (e.g., a surgeon or other medical professional) at one or more displays 116, including any tool for 3D representation of a medical environment 102. The visualization tool 114 can include a system for processing data and generating visualizations (e.g., simulations or illustrations) using a display 116.

Visualization tool 114 can be configured or designed to receive any number of different data streams from any number of data capture devices 110, sensors 104 or shape sensor systems 138 and combine them into a single data stream displayed on a display 116. The visualization tool 114 can be configured to receive a plurality of data stream components and combine the plurality of data stream components into a single data stream. For instance, the visualization tool 114 can receive a visual sensor data from one or more medical tools 112, sensors or cameras with respect to a surgical site or an area in which a surgery is performed. The visualization tool 114 can incorporate, combine or utilize multiple types of data (e.g., positioning data of a medical instrument 112 along sensor readings of pressure, temperature, vibration or any other data) to generate an output to present on a display 116. Visualization tool 114 can combine or correlate various data streams based on their respective time of generation, using for example, metadata indicative of time of each portion of data stream (e.g., timestamps in the metadata) to match the data across the data streams to use for determinations.

Display 116 can include any devices used to display data, images or information related to the medical procedure. Displays 116 can provide real-time visualizations, annotations, and measurements, such as a high-resolution monitor that shows the video stream of the procedure along with virtual annotations and measurements. A virtual annotation can refer to or include a text or voice-based annotation input by a user of system 100, such as a surgeon. The annotation can be input via an interface, and displayed on a video stream. The system 100 can use displays 116 to provide clear and accurate information to the medical team, including any updates, virtual objects 182, digital rulers 168 or any other features along with procedure video. For example, display 116 can show the alignment of the pose information with the video stream based on the compensation matrix. The display 116 can present data streams (e.g., video frames, data on events, kinematics or sensor readings) of an ongoing medical procedure (e.g., an ongoing surgery) performed using the robotic medical systems 120 as it handles, manipulates, holds or otherwise utilizes medical instruments 112 to perform surgical tasks at the surgical site.

Display 116 can show, illustrate or play data streams (e.g., instrument data 140 or video data 162) in which medical instruments 112 at or near surgical sites are shown. For example, display 116 can display a rectangular image (e.g., a frame 164 of a video data 162) of a surgical site along with at least a portion of medical instruments 112 being used to perform surgical tasks. Display 116 can provide compiled or composite images generated by the visualization tool 114 from a plurality of data capture devices 110 to provide visual feedback from one or more points of view.

Robotic medical system 120 can include any robotic systems used to perform medical procedures. Robotic medical system 120 can be communicatively coupled with a data processing system 150 or can include at least a portion of the data processing system 150. For example, robotic medical system 120 can include robotic arms and endoscopic instruments that are controlled by the holders or controllers to implement tasks of the medical procedure. The robotic medical systems 120 can utilize or employ any number of instrument systems 130 and can have their instruments systems 130 calibrated and aligned with the video stream and sensor data. Such calibrations can be maintained throughout the procedure to ensure precise and reliable performance. For example, robotic medical system 120 can execute actions using instrument systems 130 related to the medical procedure based on, or according to, real-time data from sensors and cameras for generating compensation matrices 178 to correct any out-of-calibration errors and maintain the operation within calibration thresholds 174.

The robotic medical systems 120, sometimes also referred to as the RMS 120, can include any medical robot (e.g., surgical robot) that is configured for performing medical tasks or procedures, such as by using medical instruments 112. The robotic medical systems 120 can include robotic arms for holding and maneuvering surgical instruments, one or more high-definition 3D cameras for providing views of the surgical site, and one or more consoles for allowing a user (e.g., a surgeon) to operate or maneuver the arms and tools of the robotic medical systems to perform surgeries. The robotic arms of the robotic medical systems 120 can be configured to translate movements of the user on the console or a user interface of the robotic medical systems into smaller and more accurately controlled movements of medical instruments 112 while performing the medical procedure (e.g., a medical surgery on a patient).

Instrument system 130 can include any combination of hardware and software for using, controlling or manipulating medical instruments. Instrument system 130 can include flexible bodies composed of multiple segments. For example, instrument system 130 can include a multi-segment, flexible-body endoscopic instrument. For example, instrument system 130 can include a laparoscope, bronchoscope, cystoscope, arthroscope, laparoscopic trocar, surgical robot, catheter, biopsy needle, electrosurgical unit, and a robotic arm. The robotic medical system 120 or the data processing system 150 can check, verify or ensure that the instrument systems 130 are accurately calibrated and aligned with each other based on the instrument data 140 and video data 162. Such calibrations can be maintained throughout the procedure by verifying whether variations between video and instrument data is within calibration thresholds 174, thereby ensuring precise and reliable performance. For example, instrument system 130 can include shape sensor systems 138 that process instrument data 140 to provide pose information about the tip of a particular segment 134. The error detector 172 of the data processing system 150 can use this information to detect out-of-calibration errors and trigger recalibration processes (e.g., by generating compensation matrices 178).

Flexible bodies 132 can include any combination of hardware and software for forming the structure of a multi-segment instrument system. Flexible bodies 132 can be composed of multiple segments that allow the instrument to navigate through narrow and complex paths within a patient's body. For example, flexible bodies 132 can be part of an endoscopic instrument having multiple concatenated or interconnected segments 134 used for a robotic medical procedure. Flexible bodies 132 can bend and flex to reach target areas while maintaining structural integrity. The flexibility of these bodies formed using segments 134 can be used to perform precise movements within medical procedures. For example, flexible bodies 132 can be controlled by instrument controllers 136 controlling segments 134 of the flexible body 132 based on feedback information of shape sensor system 138 to execute actions or tasks of the medical procedure based on real-time data from sensors (e.g., sensors 104 or shape sensor system 138) and cameras (e.g., data capture devices 110 images or instrument data 140).

Segments 134 can include any individual sections that make up the flexible bodies 132 of an instrument system 130. Segments 134 can be connected or coupled with each other to form a continuous and flexible structure (e.g., a flexible body 132). For example, segments 134 can be part of a multi-segment endoscopic instrument used in a robotic medical procedure. Each segment 134 can move independently, allowing the instrument system 130 to navigate through complex anatomical structures, crevices or openings within the body of a patient. The movement of the segments 134 can be controlled by instrument controllers 136 to ensure precise positioning and alignment. For example, segments 134 can include shape sensors of a shape sensor system 138 that provide continuous instrument data 140 (e.g., pose information generated from sensor data 142 or kinematics data 144) providing location of a particular portion of a particular segment 134, such as a tip of the instrument system 130, which can be used to detect out-of-calibration errors and trigger recalibration processes.

Instrument controllers 136 can include any combination of hardware and software for controlling the movement and operation of medical instruments. Instrument controllers 136 can receive and process data from various sensors and cameras to ensure accurate and precise control. For example, instrument controllers 136 can be used to manipulate a multi-segment endoscopic instrument during a robotic medical procedure. Instrument controllers 136 can execute actions related to the medical procedure based on real-time data from sensors and cameras. For example, instrument controllers 136 can apply a compensation matrix to align the pose information with the video stream, ensuring accurate calibration throughout the procedure.

Shape sensor system 138 can include any combination of hardware and software for capturing and processing data related to the shape and pose of an instrument. Shape sensor system 138 can include any type and form of sensors, including any sensor 104 or any data capture devices 110 (e.g., cameras) providing information about movement or positioning of any part of the instrument system 130. Shape sensor system 138 can provide real-time information about the position and orientation of the instrument's segments 134. Shape sensor system 138 can include fiber optic components providing information on the shape of the multi-segment instrument system 130. For example, shape sensor system 138 can be used to monitor the tip of a multi-segment endoscopic instrument during a robotic medical procedure. The data collected by various sensors (e.g., fiber optic sensors, motion sensors, tension or compression sensors, gyroscopes or velocity sensors) can be used to detect locations or positioning of any of the segments 134 of the instrument system 130 and can be used to identify or detect out-of-calibration errors (e.g., based on calibration thresholds 174) to trigger recalibration processes. For example, shape sensor system 138 can provide pose information that is aligned with the video stream using a compensation matrix 178, ensuring accurate calibration throughout the procedure.

Instrument data 140 can include any type and form of data related to the movement and operation of any portion of an instrument system 130 or any medical instruments 112, data capture devices 110 or sensors 104 that it operates or manipulates. Instrument data 140 can be collected from various sensors 104 or shape sensor system 138 as well as various data capture devices 110 or cameras that can be included within, coupled with or controlled by the instrument system 130 or the robotic medical system 120. Instrument data 140 can include timestamped data, including sensor data 142 or kinematics data 144 including timestamps, such as timestamps 166, indicating the timing at which the data was captured or recorded. For example, instrument data 140 can include data on acceleration, velocity, position, orientation or pose of a segment 134. Instrument data 140 can include or be processed or derived from various sensor data 142 and kinematics data 144. Instrument data 140 can be or include any data correspond or related to a multi-segment instrument, such as an endoscopic instrument. Instrument data 140 can be used to monitor and adjust the calibration of the instrument in real-time. For example, instrument data 140 can be processed by a data processing system to detect out-of-calibration errors and generate compensation matrices 178 for recalibration.

Instrument data 140 can include one or more streams of sensor data 142 or kinematics data 144, which can refer to or include data associated with one or more of the manipulator arms 235 or medical instruments 112 (e.g., surgical robotic tools) attached to the manipulator arms 235 of the robotic medical system 120. Such instrument data 140 can indicate various arm movements, locations or positioning (e.g., pose information). Data corresponding to medical instruments 112 can be captured or detected by one or more displacement transducers, orientational sensors, positional sensors, or other types of sensors and devices to measure parameters or generate kinematics information. The kinematics data 144 can include sensor data along with time stamps and an indication of the medical instrument 112 or type of medical instrument 112.

Sensor data 142 can include any type and form of data collected from sensors used in a medical procedure, including sensors 104 or shape sensor system 138. Sensor data 142 can include readings, measurements, recordings or signals of any sensor 104, whether deployed within the robotic medical system 120, within the instrument system 130 or outside of the robotic medical system 120 or instrument system 130 within the medical environment 102. Sensor data 142 can include data indicating or providing information about the position, movement, and status of an instrument system 130, its medical instruments 112 (e.g., medical instruments being handled by the robotic medical system 120 or instrument system 130) and other objects 106. For example, sensor data 142 can include information on pose (e.g., the pose information) based on shape sensors of the shape sensors 104 of the shape sensor system 138 that are attached to, integrated within, or otherwise measuring or monitoring a multi-segment endoscopic instrument system 130. Sensor data 142 can be used to compare against various calibration thresholds 174 to detect out-of-calibration errors and trigger recalibration processes (e.g., generation of compensation matrices 178 to apply by instrument controllers 136 with instrument systems 130). For example, sensor data 142 can be aligned with the video stream using a compensation matrix to ensure accurate calibration throughout the procedure.

Kinematics data 144 can include any type and form of data related to the movement and mechanics of segments 134 of the instrument system 130 or medical instruments 112 handled or operated by the instrument system 130. Kinematics data 144 can provide information about the position, velocity, and acceleration of different segments 134 of the flexible bodies 132 of instrument systems 130. For example, kinematics data 144 can be collected from sensors 104 or data capture devices 110 included within, coupled with or attached to a multi-segment endoscopic instrument during a robotic medical procedure. Kinematics data 144 can be used to detect out-of-calibration errors and trigger recalibration processes (e.g., generation of compensation matrices 178 to apply by instrument controllers 136 with instrument systems 130). For example, kinematics data 144 can be aligned with the pose information and video stream using a compensation matrix 178 to ensure accurate calibration throughout the procedure.

Data processing system 150 can include any combination of hardware and software for processing and analyzing data related to a medical procedure. Data processing system 150 can receive and process data from various sensors 104 or data capture devices 110 of instrument systems 130 or their medical instruments 112. For example, data processing system 150 can be used to detect out-of-calibration errors and generate compensation matrices 178 for recalibration of the sensor and video data. This system can also provide real-time feedback and guidance to the medical team. For example, data processing system 150 can include vision system functions 160, calibrators 170, and visualization functions 180 to ensure accurate and precise calibration of medical instruments throughout the procedure and provide virtual objects 182 for the purposes of the procedure.

The data processing system 150, as well as any of its components or functionalities can be served or provided in using any one or more technologies. For instance, data processing system 150 or its components can a part of or include a cloud computing environment functionality or features or include a group of logically grouped servers implemented via various distributed computing techniques. The logical group of servers may be referred to as a data center, server farm or a machine farm. The servers can be centered within data center or geographically dispersed. A data center or machine farm may be administered as a single entity, or the machine farm can include a plurality of machine farms. The servers within each machine farm can be heterogeneous—one or more of the servers or machines can operate according to one or more type of operating system platform.

The data processing system 150, or components thereof, can be located at least partially at the location of the surgical facility associated with the medical environment 102 or remotely therefrom. Elements of the data processing system 150, or components thereof can be accessible via portable client devices, such as laptops, mobile devices, wearable smart devices, etc. The data processing system 150, or components thereof, can include other or additional elements that can be considered desirable to have in performing the functions described herein. The data processing system 150, or components thereof, can include, or be associated with, one or more components or functionality of a computing including, for example, one or more processors 310 coupled with memory (e.g., 315 or 325) that can store instructions, data or commands for implementing the functionalities of the data processing system 150 discussed herein.

Vision system function 160 can include any combination of hardware and software for receiving, gathering, and processing image data related to a medical procedure. Video system function 160 can be coupled with various data capture devices 110 (e.g., cameras) or sensors 104 of various types and from various medical instruments 112 or instrument systems 130 to gather or provide video data 162. Vision system function 160 can provide video frames 164 that are timestamped using timestamps 166 for real-time visualizations and measurements to the medical team. For example, vision system function 160 can include cameras that provide a video stream of the procedure along with timestamps 166 associated with each frame 164 of the video data 162 within the stream. Vision system function 160 can include functionality for generating and displaying digital rulers 168 for measuring dimensions of anatomical structures. The digital ruler 168 can be overlayed on an image frame depicting an anatomical structure. Vision system function 160 can detect out-of-calibration errors based on the video stream and generate compensation matrices 178 for recalibration.

Video data 162 can include any type and form of image data including one or more images or frames 164 of a video stream captured during a medical procedure. Video data 162 can be collected from cameras or other imaging devices within the medical environment 102. For example, video data 162 can include a continuous video stream that provides real-time visual information about the procedure. Video data 162 can be used to monitor and analyze the procedure, ensuring accurate and precise execution. For example, video data 162 can be processed to detect out-of-calibration errors between the video stream and the pose information from the shape sensor system. The instrument system 130 can then apply a compensation matrix 178 generated responsive to the out-of-calibration error to align the video data 162 with the instrument data 140 (e.g., the pose information from the sensors 104) to maintain calibration throughout the procedure.

Video data 162 can include one or more video streams having any sequence of media, including images or video frames or clips. Video stream 162 can include images or videos captured by a medical instruments 112 (e.g., endoscopic camera) can be sent to the visualization tool 114. The robotic medical system 120 can include one or more input ports to receive video stream with frames 164 via direct or indirect connection of one or more auxiliary devices. For example, the visualization tool 114 can be connected to the robotic medical systems 120 to receive the images from the medical instrument 112 when the medical instrument 112 is installed in the robotic medical systems 120 (e.g., on a manipulator arm of the robotic medical systems 120 that is used for moving, managing or otherwise handing medical instruments 112). The visualization tool 114 can combine the instrument data 140 or the video data 162 from the data capture devices 110 or sensors 104 or shape sensor system 138 and the medical tool 112 into a single combined data stream to be provided to the vision system function 160 or calibrator 170.

Frames 164 can include individual images or snapshots that make up the video data 162. The video frames 164 can be captured at regular time intervals to create a continuous video stream, such as 30, 45, 60, 90, 120, 240 or more than 240 frames per second. For example, frames 164 can include images of the surgical site captured by a camera during a robotic medical procedure. Each frame 164 can provide detailed visual information about a location, position or pose of a flexible body 132 of an instrument system 130, which can be used to monitor and analyze the procedure. For example, frames 164 can include images of a shape of the flexible body 132 of the instrument system 130 that can be compared with the pose information from the shape sensor system 138 to detect spatial mismatches between the two data and determine the presence of the out-of-calibration errors (e.g., spatial calibration error). The system can then apply a compensation matrix 178 to align temporally and spatially the frames 164 with the pose information to satisfy the calibration thresholds 174, ensuring accurate calibration.

Timestamps 166 can include any type and form of data identifying or corresponding to time associated with the frames 164 of the video data 162. The timestamps 166 can indicate the exact time at which each frame 164 was captured. For example, timestamps 166 can be used to synchronize the video data 162 with other data streams, such as instrument data 140 identifying the pose information from the shape sensor system 138. This synchronization can be used to maintain accurate temporal (e.g., time-related) calibration between different data during the procedure. Timestamps 166 associated with instrument data 140 and video data 162 can be used to identify temporal (e.g., time-based) out-of-calibration errors. For example, the system can apply a compensation matrix 178 to adjust, offset or align the timestamps 166 of the frames 164 with the timestamps 166 of the instrument data 140 indicating the pose information, thereby ensuring precise temporal calibration throughout the procedure.

Timestamps 166 can be used for temporal adjustment or calibration of data using time offsets. For instance, timestamps 166 of one data stream can be adjusted temporally with respect to the readings of another data stream using one or more offsets used to align the time difference between the two data streams. The offsets can be applied to a first or a second of the two sets of timestamps 166 in order to offset the timestamps 166 of one of the data streams with respect to the timestamps 166 of the other. In doing so, the offsets can be used to temporally align any type of data, such as video data of an instrument point with sensor data or kinematics data of the same instrument point.

Digital ruler 168 can include any tools or software used to measure dimensions of anatomical structures during a medical procedure. This tool can provide real-time measurements that can be displayed via a display 116. For example, digital ruler 168 can be used to measure the size of a tumor or the length of a blood vessel during a robotic medical procedure. These measurements can be used for planning and executing various tasks during the procedure. For example, the data processing system 150 can activate the digital ruler 168 after applying a compensation matrix 178 to ensure accurate calibration. The measurements made using the digital ruler 168 can then be displayed to the medical team, providing valuable information for the procedure.

Calibrator 170 can include any combination of hardware and software for detecting and correcting out-of-calibration errors during a medical procedure. The calibrator 170 can process data from various sensors and cameras to ensure accurate calibration. The calibrator 170 can operate the error detector 172 to compare instrument data 140 with video data 162, including any sensor data 142 or kinematics data 144 indicative of the movement or positioning of one or more segments 134 of the instrument system 130 and video frames 164 of the same one or more segments 134. The calibrator 170 can compare the timestamps 166 of the instrument data 140 and the video frames 164 and check if the spatial mismatches exceed calibration thresholds 174. In response to the spatial mismatches between the two or more data streams (e.g., 140 and 162) or temporal mismatches between the two or more data streams (e.g., 140 and 162) exceeding calibration thresholds 174, the calibrator 170 can detect an out-of-calibration error and administer a recalibration procedure. For example, calibrator 170 can detect out-of-calibration errors between the video data and the pose information from the shape sensor system 138, such as a spatial out-of-calibration error or a temporal out-of-calibration error. The calibrator 170 can then generate and apply a compensation matrix 178 to realign the data streams. For example, the calibrator 170 can apply a compensation matrix 178 for a spatial alignment, or apply a compensation matrix 178 for a temporal alignment, in order to realign the data streams or the spatial or temporal information of frames of the data streams. For example, calibrator 170 can include and utilize error detectors 172 and calibration thresholds 174 to identify and correct calibration errors in real-time, maintaining precise calibration throughout the procedure.

Error detector 172 can include any devices or software used to detect out-of-calibration errors during a medical procedure. This component can analyze data from various sensors and cameras to identify discrepancies. For example, error detector 172 can compare the video data 162 with the pose information generated based on sensor data 142 from the shape sensor system 138 to detect an out-of-calibration error. For example, the out-of-calibration error can be a spatial out-of-calibration error, such as a mismatch in location or positioning of the portion of the instrument system 130 as reflected by the instrument data 140 (e.g., sensor data 142 or kinematics data 144) indicating the pose information and by the video data 162 (e.g., frames 164). For example, the out-of-calibration error can be a temporal out-of-calibration error, such as a mismatch in timing between the movement or positioning of the instrument system 130 as reflected by the instrument data 140 and the video data 162. In response to the detection of the out-of-calibration error by the error detector 172, the calibrator 170 can trigger recalibration process to correct such errors. For example, error detector 172 can use calibration thresholds to determine when an error has occurred and the calibrator 170 can, responsive to such determination, initiate the generation of a compensation matrix 178 to realign the data streams.

Calibration thresholds 174 can include any predefined values or criteria used to determine when recalibration is necessary during a medical procedure. Calibration thresholds 174 can include any temporal or spatial limitations that can be used to compare the size of the temporal or spatial mismatch between different data (e.g., instrument data 140 and video data 162) to identify or detect out-of-calibration errors. Calibration thresholds 174 can include a temporal thresholds corresponding to a time duration limitations (e.g., of the instrument system 130 as reflected by two different data streams) for indicating or detecting temporal out-of-calibration errors. Calibration thresholds 174 can include spatial thresholds corresponding to spatial or distance limitations (e.g., of the instrument system 130 as reflected by two different data streams) for indication or detecting spatial out-of-calibration errors.

Calibration thresholds 174 can be based on various factors, such as the type of task being performed or the accuracy required. For instance, a type of a task being implemented can be detected and calibrations can be done according to the type of task detected or identified. The calibration thresholds 174 can include threshold range of movements along a particular path, based on the type of a task or a type of a medical procedure. For instance, the calibration thresholds 174 can include a range of allowed movements along any combination of length, width or height, allowing for free movement within the range for a particular task (e.g., an action performed within a particular anatomical structure of a body). When a user (e.g., a surgeon) moves an instrument outside of the calibration threshold range, the data processing system 130 can generate a compensation matrix 178 to make the correction. For example, there can be a first set of calibration thresholds 174 for a first task of a plurality of tasks of a medical procedure and a second set of calibration threshold 174 for a second task of the plurality of tasks of the same medical procedure. Compensation matrix 178 can be then generated for a given task of a medical procedure, responsive to the exceeding of the calibration thresholds 174 for that task. For example, there can be a first set of calibration thresholds 174 for a medical procedure a second set of calibration threshold 174 for a second medical procedure. Each set of calibration thresholds 174 can cover one or more tasks or actions implemented during each medical procedure. Compensation matrices 178 can be generated for a given task of a medical procedure, responsive to the exceeding of the calibration thresholds 174 for that medical procedure. For example, calibration thresholds 174 can be used to detect out-of-calibration errors between the video data and the pose information from the shape sensor system. When an error exceeds the calibration threshold 174, the calibrator 170 can generate and apply a compensation matrix 178 to adjust the two data streams spatially or temporally and thereby recalibrate the system. For example, calibration thresholds 174 can be adjusted based on the specific requirements of the procedure to ensure precise and reliable calibration.

Factory matrices 176 can include any one or more matrices of values or settings for setting up temporal or spatial offsets between different data streams. Factory matrices 176 can include a matrix including predefined calibration data used to initially calibrate the sensors and cameras before a medical procedure. A factory matrix 176 can include or provide settings or values that are a baseline for accurate calibration between the instrument data 140 and the video data 162. For example, factory matrices 176 can be used to align the pose information of a segment 134 of an instrument system 130 from the shape sensor system 138 with the video data 162 from the cameras capturing the same segment 134. This initial calibration can be used for ensuring accurate data collection during the procedure at an initial stage (e.g., before any recalibrations using compensation matrices 178. For example, factory matrices 176 can be different from the compensation matrices 178 used or generated during the procedure to correct out-of-calibration errors in real-time.

Compensation matrices 178 can include any one or more matrices of values or settings for adjusting or changing temporal or spatial offsets between different data streams from those of the factory matrices 176. For instance, a compensation matrix 178 can include data used to correct out-of-calibration errors during a medical procedure. Compensation matrices 178 can be generated in real-time based on data from various sensors 104 and cameras. For example, compensation matrices 178 can be used to align the pose information generated from the instrument data 140 of a shape sensor system 138 with the video data 162 from the cameras. This realignment can ensure accurate calibration throughout the procedure. For example, compensation matrices 178 can be applied by the instrument controllers 136 to execute actions related to the medical procedure based on the aligned data streams.

Compensation matrix 178 can include any arrangement or array of values organized in rows, columns or both rows and columns. The values of the matrix or array can be arranged to provide a particular transformation of an image or data. The compensation matrix 178 can be configured to provide spatial synchronization or alignment between data of different data streams. For example, a first compensation matrix 178 can include adjustment values for spatially aligning sensor data or readings of an instrument point with video data of the same instrument point. For example, a second compensation matrix 178 can include rows and columns of adjustment values for spatially aligning kinematics data or readings of an instrument point with video data of the same instrument point. For instance, a compensation matrix 178 can be arranged or configured to provide a rigid transform, which can be also known as a Euclidean transformation or Euclidean isometry. The rigid transformation can be a geometric transformation that preserves the Euclidean distance between every pair of points. Thus, the shape and size of an object that is transformed can remain unchanged after the transformation. A rigid transformation can include one or more rotations, translations, reflections, or any sequence of these arrangements in which the size and shape of the object remain unchanged. In three-dimensional space, every rigid motion can be decomposed into a combination of a rotation and a translation, sometimes referred to as a roto-translation.

The compensation matrix 178 can be used as a part of an interactive parametric model allowing users to adjust parameters and see the effect of the modeling. An interactive parametric model can include a functionality to dynamically adjust parameters based on real-time data inputs or user interactions. An interactive parametric model can be configured based on the state of a tool coupled with an instrument system and can allow for calibration and alignment of various data streams, such as pose information from a shape sensor system and video streams from a vision system. By detecting out-of-calibration errors and applying compensation matrices, the interactive parametric model can maintain the accuracy of the data within a particular range to ensure synchronization throughout the procedure. The interactive parametric model can provide real-time adaptability to improve the accuracy and effectiveness of the medical procedure. The calibration can utilize the interactive parametric model configured based on a state of a tool (e.g., instrument 112) that is coupled with the robotic medical system 120. The state of the tool can include state information about an instrument, such as that a tool (e.g., instrument) is plugged in, engaged, disengaged, initiated, calibrated or uncalibrated. For example, a compensation matrix 178 can provide transformations such as hand-eye calibration, which can align the shape sensor tip with the camera. For example, a compensation matrix 178 can provide a trajectory alignment, which can adjust the pose information and video data to ensure accurate calibration during medical procedures. For example, the compensation matrix 178 can be used to recalibrate the positioning of robotic instrument tips, such as forceps, to maintain precise alignment and functionality of the instrument.

Visualization function 180 can include any tools or software used to create visual representations of data related to a medical procedure. Visualization function 180 can provide real-time visualizations and annotations to the medical team. For example, visualization function 180 can generate virtual objects 182 or annotations based on the calibrated pose information and video data. These visualizations can enhance the precision and efficiency of the procedure. For example, visualization function 180 can display virtual annotations with the video stream to provide clear and accurate information to the medical team.

Virtual objects 182 can include any digital representations of anatomical structures or other relevant features during a medical procedure. Virtual objects 182 can be generated based on data from various sensors and cameras. For example, virtual objects 182 can include 3D models of anatomical structures that are displayed with the video stream. Virtual objects 182 can provide valuable information for planning and executing the procedure. For example, virtual objects 182 can be registered based on the calibrated pose information and video data using a compensation matrix 178.

Various data, transmissions or network traffic, such as transmissions communicated between the data processing system 150 and the robotic medical system 120, can be implemented via one or more networks 101. A network 101 can include any type or form of a communication link, medium or a network for providing or facilitating signal communications. A network can include a wire, a cable or a line, as well as a wireless link (e.g., Bluetooth, Near-Field Communications or Wi-Fi). The geographical scope of the network 101 can vary widely and can include a body area network (BAN), a personal area network (PAN), a local-area network (LAN) (e.g., Intranet), a metropolitan area network (MAN), a wide area network (WAN), or the Internet. The topology of the network 101 can assume any form such as point-to-point, bus, star, ring, mesh, tree, etc. The network 101 can utilize different techniques and layers or stacks of protocols, including, for example, intra-system communication protocols for communications between devices within a single enclosure or a system or protocols for device-to-device communications, including Ethernet protocol, the internet protocol suite (TCP/IP), the ATM (Asynchronous Transfer Mode) technique, the SONET (Synchronous Optical Networking) protocol, the SDH (Synchronous Digital Hierarchy) protocol, etc. The TCP/IP internet protocol suite can include application layer, transport layer, internet layer (including, e.g., IPv6), or the link layer. The network 101 can be a type of a broadcast network, a telecommunications network, a data communication network, a computer network, a Bluetooth network, or other types of wired and wireless mediums, connections, links or networks.

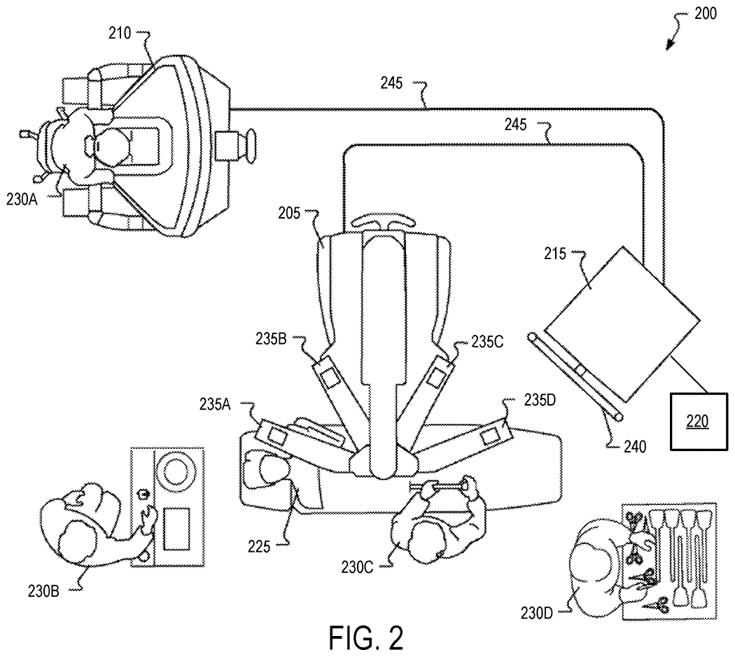

FIG. 2 depicts a surgical system 200, in accordance with some embodiments. The surgical system 200 may be an example of the medical environment 102. The surgical system 200 may include a robotic medical system 205 (e.g., the robotic medical system 120), a user control system 210, and an auxiliary system 215 communicatively coupled one to another. A visualization tool 220 (e.g., the visualization tool 114) may be connected to the auxiliary system 215, which in turn may be connected to the robotic medical system 205. Thus, when the visualization tool 220 is connected to the auxiliary system 215 and this auxiliary system is connected to the robotic medical system 205, the visualization tool may be considered connected to the robotic medical system. In some embodiments, the visualization tool 220 may additionally or alternatively be directly connected to the robotic medical system 205.

The surgical system 200 may be used to perform a computer-assisted medical procedure on a patient 225. In some embodiments, surgical team may include a surgeon 230A and additional medical personnel 230B-230D such as a medical assistant, nurse, and anesthesiologist, and other suitable team members who may assist with the surgical procedure or medical session. The medical session may include the surgical procedure being performed on the patient 225, as well as any pre-operative (e.g., which may include setup of the surgical system 200, including preparation of the patient 225 for the procedure), and post-operative (e.g., which may include clean up or post care of the patient), or other processes during the medical session. Although described in the context of a surgical procedure, the surgical system 200 may be implemented in a non-surgical procedure, or other types of medical procedures or diagnostics that may benefit from the accuracy and convenience of the surgical system.

The robotic medical system 205 can include a plurality of manipulator arms 235A-235D to which a plurality of medical tools (e.g., the medical tool 112) can be coupled or installed. Each medical tool can be any suitable surgical tool (e.g., a tool having tissue-interaction functions), imaging device (e.g., an endoscope, an ultrasound tool, etc.), sensing instrument (e.g., a force-sensing surgical instrument), diagnostic instrument, or other suitable instrument that can be used for a computer-assisted surgical procedure on the patient 225 (e.g., by being at least partially inserted into the patient and manipulated to perform a computer-assisted surgical procedure on the patient). Although the robotic medical system 205 is shown as including four manipulator arms (e.g., the manipulator arms 235A-235D), in other embodiments, the robotic medical system can include greater than or fewer than four manipulator arms. Further, not all manipulator arms can have a medical tool installed thereto at all times of the medical session. Moreover, in some embodiments, a medical tool installed on a manipulator arm can be replaced with another medical tool as suitable.

One or more of the manipulator arms 235A-235D and/or the medical tools attached to manipulator arms can include one or more displacement transducers, orientational sensors, positional sensors, and/or other types of sensors and devices to measure parameters and/or generate kinematics information. One or more components of the surgical system 200 can be configured to use the measured parameters and/or the kinematics information to track (e.g., determine poses of) and/or control the medical tools, as well as anything connected to the medical tools and/or the manipulator arms 235A-235D.

The user control system 210 can be used by the surgeon 230A to control (e.g., move) one or more of the manipulator arms 235A-235D and/or the medical tools connected to the manipulator arms. To facilitate control of the manipulator arms 235A-235D and track progression of the medical session, the user control system 210 can include a display (e.g., the display 116) that can provide the surgeon 230A with imagery (e.g., high-definition 3D imagery) of a surgical site associated with the patient 225 as captured by a medical tool (e.g., the medical tool 112, which can be an endoscope) installed to one of the manipulator arms 235A-235D. The user control system 210 can include a stereo viewer having two or more displays where stereoscopic images of a surgical site associated with the patient 225 and generated by a stereoscopic imaging system can be viewed by the surgeon 230A. In some embodiments, the user control system 210 can also receive images from the auxiliary system 215 and the visualization tool 220.

The surgeon 230A can use the imagery displayed by the user control system 210 to perform one or more procedures with one or more medical tools attached to the manipulator arms 235A-235D. To facilitate control of the manipulator arms 235A-235D and/or the medical tools installed thereto, the user control system 210 can include a set of controls. These controls can be manipulated by the surgeon 230A to control movement of the manipulator arms 235A-235D and/or the medical tools installed thereto. The controls can be configured to detect a wide variety of hand, wrist, and finger movements by the surgeon 230A to allow the surgeon to intuitively perform a procedure on the patient 225 using one or more medical tools installed to the manipulator arms 235A-235D.

The auxiliary system 215 can include one or more computing devices configured to perform processing operations within the surgical system 200. For example, the one or more computing devices can control and/or coordinate operations performed by various other components (e.g., the robotic medical system 205, the user control system 210) of the surgical system 200. A computing device included in the user control system 210 can transmit instructions to the robotic medical system 205 by way of the one or more computing devices of the auxiliary system 215. The auxiliary system 215 can receive and process image data representative of imagery captured by one or more imaging devices (e.g., medical tools) attached to the robotic medical system 205, as well as other data stream sources received from the visualization tool. For example, one or more image capture devices (e.g., the image capture devices) can be located within the surgical system 200. These image capture devices can capture images from various viewpoints within the surgical system 200. These images (e.g., video streams) can be transmitted to the visualization tool 220, which can then passthrough those images to the auxiliary system 215 as a single combined data stream. The auxiliary system 215 can then transmit the single video stream (including any data stream received from the medical tool(s) of the robotic medical system 205) to present on a display (e.g., the display 116) of the user control system 210.

In some embodiments, the auxiliary system 215 can be configured to present visual content (e.g., the single combined data stream) to other team members (e.g., the medical personnel 230B-230D) who might not have access to the user control system 210. Thus, the auxiliary system 215 can include a display 240 configured to display one or more user interfaces, such as images of the surgical site, information associated with the patient 225 and/or the surgical procedure, and/or any other visual content (e.g., the single combined data stream). In some embodiments, display 240 can be a touchscreen display and/or include other features to allow the medical personnel 230A-230D to interact with the auxiliary system 215.

The robotic medical system 205, the user control system 210, and the auxiliary system 215 can be communicatively coupled one to another in any suitable manner. For example, in some embodiments, the robotic medical system 205, the user control system 210, and the auxiliary system 215 can be communicatively coupled by way of control lines 245, which can represent any wired or wireless communication link that can serve a particular implementation. Thus, the robotic medical system 205, the user control system 210, and the auxiliary system 215 can each include one or more wired or wireless communication interfaces, such as one or more local area network interfaces, Wi-Fi network interfaces, cellular interfaces, etc. It is to be understood that the surgical system 200 can include other or additional components or elements that can be needed or considered desirable to have for the medical session for which the surgical system is being used.

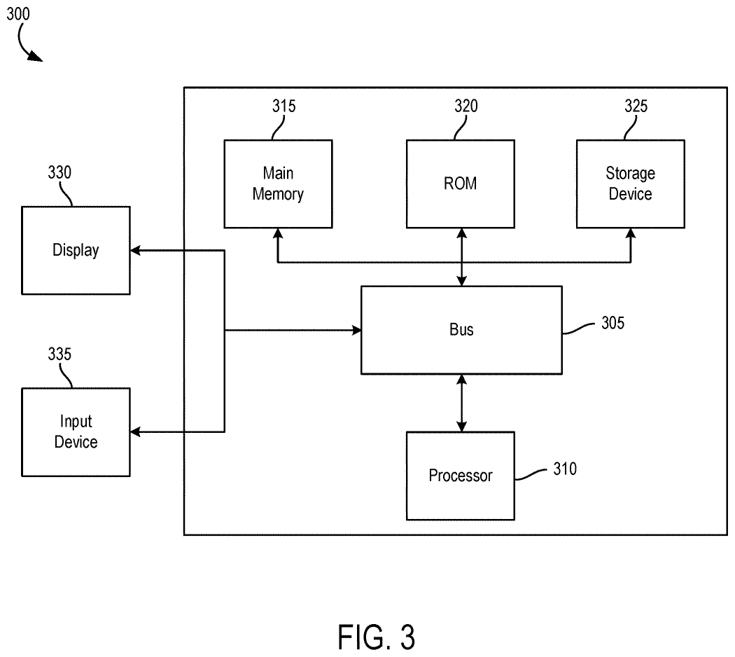

FIG. 3 depicts an example block diagram of an example computer system 300 is shown, in accordance with some embodiments. The computer system 300 can be any computing device used herein and can include or be used to implement a data processing system or its components. The computer system 300 includes at least one bus 305 or other communication component or interface for communicating information between various elements of the computer system. The computer system further includes at least one processor 310 or processing circuit coupled to the bus 305 for processing information. The computer system 300 also includes at least one main memory 315, such as a random-access memory (RAM) or other dynamic storage device, coupled to the bus 305 for storing information, and instructions to be executed by the processor 310. The main memory 315 can be used for storing information during execution of instructions by the processor 310. The computer system 300 can further include at least one read only memory (ROM) 320 or other static storage device coupled to the bus 305 for storing static information and instructions for the processor 310. A storage device 325, such as a solid-state device, magnetic disk or optical disk, can be coupled to the bus 305 to persistently store information and instructions.

The computer system 300 can be coupled via the bus 305 to a display 330, such as a liquid crystal display, or active-matrix display, for displaying information. An input device 335, such as a keyboard or voice interface can be coupled to the bus 305 for communicating information and commands to the processor 310. The input device 335 can include a touch screen display (e.g., the display 330). The input device 335 can also include a cursor control, such as a mouse, a trackball, or cursor direction keys, for communicating direction information and command selections to the processor 310 and for controlling cursor movement on the display 330.

The processes, systems and methods described herein can be implemented by the computer system 300 in response to the processor 310 executing an arrangement of instructions contained in the main memory 315. Such instructions can be read into the main memory 315 from another computer-readable medium, such as the storage device 325. Execution of the arrangement of instructions contained in the main memory 315 can cause the processor 310 or the computer system 300 as a whole to perform the illustrative functionalities or processes described herein. One or more processors in a multi-processing arrangement can also be employed to execute the instructions contained in the main memory 315. Hard-wired circuitry can be used in place of or in combination with software instructions together with the systems and methods described herein. Systems and methods described herein are not limited to any specific combination of hardware circuitry and software.

Technical solutions can overcome the issue of sensor synchronization and alignment in digital features and robotics, such as in endoscopic procedures. In some configurations, the technical solutions can include aligning a shape sensor providing positional information with a camera to accurately reflect movements. Challenges can arise from spatial alignment issues caused by factors like heat conditions or impacts, and synchronization issues due to clock drift over time. For example, initial factory calibration can be disrupted by these factors (e.g., impact, pressure or heat condition based issues) leading to misalignment and synchronization problems. The technical solutions can address these challenges by providing methods for synchronization drift correction and sensor calibration, providing accurate and reliable performance during medical procedures.

Technical solutions can include, for example, systems and methods to improve precision in controlling robotic instruments during procedures by compensating for time latency and ensuring accurate spatial positioning. In some configurations, the technical solutions can include real-time surgical navigation by spatially calibrating preoperative imaging with real-time endoscopic views. For example, this can enhance navigation and allow for precise overlay of imaging data. The technical solutions can include automated surgical task execution, where robotic systems can autonomously perform tasks like suturing and cutting with reduced latency and precise spatial calibration. These methods can improve the accuracy and efficiency of robotic medical procedures.

Technical solutions can include, for example, automatically recalibrating the spatial positions of sensors relative to one another during day-to-day operations, functioning autonomously and unsupervised. In some configurations, the technical solutions can include maintaining temporal synchronization to ensure consistent performance and accuracy in endoscopic procedures. For example, the technology can employ a robust algorithm to align timestamps and reference frames of the endoscope's camera and additional sensors, such as shape sensors. This method can correct synchronization drift and ensure accurate sensor calibration, enhancing the overall precision of the medical procedure.

Technical solutions can include, for example, hand-eye calibration that relies on multiple constraints and operates autonomously without operator supervision. In some configurations, the technical solutions can include visual odometry methods adapted for medical imaging to extract the camera's trajectory. For example, the system can combine measurements from shape sensors, other sensors, and the robot's kinematics to refine and correct calibration discrepancies. The joint optimization process can resolve time-synchronization issues between clocks, ensuring seamless operation and enhanced accuracy. These design constraints can ensure that the calibration process is robust and reliable.

Technical solutions can include, for example, a system using multiple sensors to address hand-eye calibration involving time-related issues in surgical environment (e.g., in the wild calibration). In some configurations, the technical solutions can include operating on robotic systems autonomously on real-world data without requiring intervention from experts or operators. For example, the system can incorporate multiple sensors and kinematics, to improve the accuracy and efficiency of robotic medical procedures.

Technical solutions can include, for example, addressing the limitations of various systems. In some configurations, the technical solutions can include automatic calibration refinement to address factors like heat conditions or impacts. For example, the system can ensure synchronization between shape sensors and kinematics, improving the overall performance of the robotic system. These improvements can enhance the accuracy and reliability of the system, providing better outcomes for medical procedures.

Technical solutions can include, for example, improving accuracy in reconstructing to scale and enhancing navigation without a predefined map. In some configurations, the technical solutions can include supporting pulmonary interventions and various long-term diseases. For example, the system can enable new procedures without a CT scan by using shape sensing to build the map live. These applications can significantly enhance the capabilities of robotic medical systems, providing better precision and efficiency in various medical interventions.

Technical solutions can include, for example, three stages of calibration: factory calibration, homing, and in-the-wild calibration. In some configurations, the technical solutions can include factory calibration involving detection and tracking of calibration targets, camera pose estimation, forward kinematics, shape sensor pose, streams time alignment, and hand-eye calibration. For example, the system can perform real-time calibration during the procedure, involving trajectory subset acquisition, rigid transform compensation per frame, and hand-eye calibration. These stages of calibration can ensure accurate and reliable performance throughout the medical procedure.

Technical solutions can include, for example, factory calibration involving the detection and tracking of calibration targets and camera pose estimation. In some configurations, the technical solutions can include forward kinematics and shape sensor pose alignment. For example, the system can perform time synchronization to ensure accurate calibration. The method can involve moving the endoscope without actuating the robot joints, recording images of a known calibration object, and performing time offset alignment between the shape sensor data and the camera images. This process can ensure synchronization and accurate calibration of the system.

Technical solutions can provide homing calibration, which can include a process to align and synchronize the spatial positions and timestamps of sensors and robotic components before the start of a medical procedure or a task. The homing calibration, which can include a board-enhanced calibration, can allow for the sensors, such as shape sensors and cameras, to be accurately aligned with the robot's kinematics, providing precise and reliable data during the procedure. For example, during the homing calibration process, a fixed calibration object or a physical marker, such as a carved checkerboard, is placed in a known position relative to the robot. This can be implemented in a workspace or using a virtual object on the robot's display screen. For instance, a homing calibration process can utilize or rely on one or more rendered calibration targets to perform the recalibration during the startup of the robot system. The robot can capture an image of this object and compare it with its known kinematic data, allowing for real-time hand-eye calibration and time offset correction. This can allow for the robot-camera alignment to be precise at the start of each session, improving the accuracy and performance of the robotic system during medical procedures.

Technical solutions can provide in-the-wild autocalibration, which can include a process to automatically recalibrate the spatial positions and synchronize the timestamps of sensors and robotic components during the performance of a medical procedure or task. The in-the-wild autocalibration can allow for the sensors, such as shape sensors and cameras, to remain accurately aligned with the robot's kinematics in real-time, providing precise and reliable data throughout the procedure. For example, during an endoscopic procedure, the system can continuously monitor the alignment of the shape sensor and camera. If an out-of-calibration error is detected, the system can apply a compensation matrix to realign the data, ensuring that the instrument's position and orientation are accurately reflected in the video stream. This can maintain the precision and effectiveness of the robotic system, even as conditions change during the procedure.

During an in-the-wild autocalibration, a rigidity selection test can be performed to determine or select the frames to use for extracting the camera pose and perform the autocalibration. Doing so can improve the autocalibration by removing the unknowns, such as tissue deformation during the procedure. Camera pose estimation can involve capturing and processing images to determine the camera's position and orientation relative to the instrument system. Forward kinematics can be used to predict the expected position and orientation of the instrument based on the movement data of its segments. Shape sensor pose can involve capturing and processing data from shape sensors attached to the instrument system to determine its actual pose. Streams time alignment can ensure that the timestamps of different data streams, such as video and sensor data, are synchronized for accurate calibration. Hand-eye calibration can align the spatial information from the camera and sensors with the robot's kinematics to ensure precise control. Adaptive online estimation can continuously refine calibration parameters in real-time to maintain accuracy during the procedure.

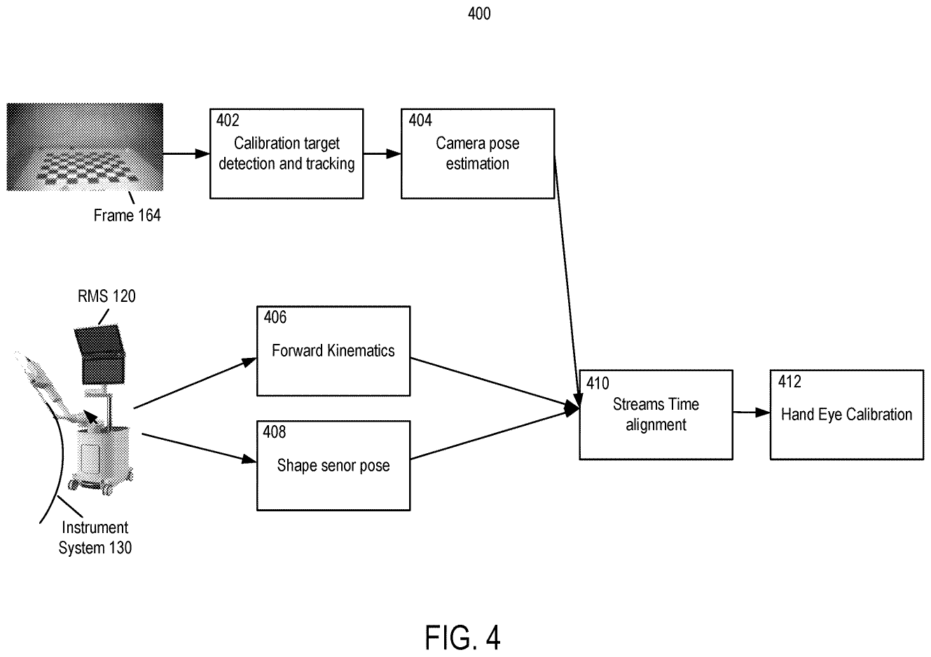

FIG. 4 depicts an example flow diagram of a method 400 for providing a factory calibration of the instrument system. The method 400 can be implemented using, for example, system 100 depicted in FIG. 1 or the computing system 300 depicted in FIG. 3. The method 400 can include acts 402-412 implementing various operations of the spatiotemporal autocalibration of an instrument system 130, such as an endoscope used in a medical procedure.

At act 402, the method can include a data processing system identifying or receiving one or more frames 164 to be used for performing calibration target detection and tracking. At 402, the method can include identifying and tracking specific calibration targets (e.g., for a factory calibration) to establish a reference for calibration. The reference can include a calibration board reference point provided in a camera coordinate system.

At act 404, the method can implement camera pose estimation based on the frame 164. The camera pose estimation can be implemented as a part of a factory calibration. At 404, the method can include the system can utilize the frame 164 to determine the pose of the particular segment of the instrument system. For instance, a calibration board can be used to estimate the camera tip position relative to the calibration board or some other fixed reference point. The pose information can be determined based on analysis of the image, using for example, machine learning to identify the segment feature and identify the segment pose or shape with respect to the overall multi-segment system. For instance, the estimation of the camera pose can be implemented by extracting a tracked corner of the calibration target and back projecting this point based on prior knowledge of the board dimensions to determine one or more 3D locations.

At act 406, the robotic medical system can provide or forward kinematics data to the data processing system for processing. The kinematics data can be used to determine the expected position and orientation of the instrument based on the movement data of its segments. The kinematics data can be used in predicting the endoscope's pose and aligning it with the actual sensor data.

At act 408, the robotic medical system can provide or identify the shape sensor pose. For instance, the method can capture and process the data from the shape sensors attached to the instrument system (e.g., endoscope) to determine its actual pose. The shape sensor pose data can then be compared with the forward kinematics data to detect any discrepancies.

At act 410, the method can include determinations of streams time alignment. The method can, for example, compare or check the synchronization of the timestamps of the video data and the sensor data. This can allow for the data from different sources to be accurately aligned in time, allowing for improved calibration.

At act 412, the method can perform the hand-eye calibration. The method can include aligning the endoscope's pose with the camera's view to ensure that the visual data accurately reflects the endoscope's movements. This can be done, for example, using a factory matrix providing values or settings for synchronization of the instrument data and the video data. The hand-eye calibration can allow for spatial and temporal alignment in a calibrated manner.

The method described in FIG. 4 can facilitate the endoscope being or remaining accurately calibrated during the medical procedure, providing precise and reliable data for the medical team. This method addresses the challenges of spatial and temporal alignment, ensuring that the endoscope's movements are accurately reflected in the video data.

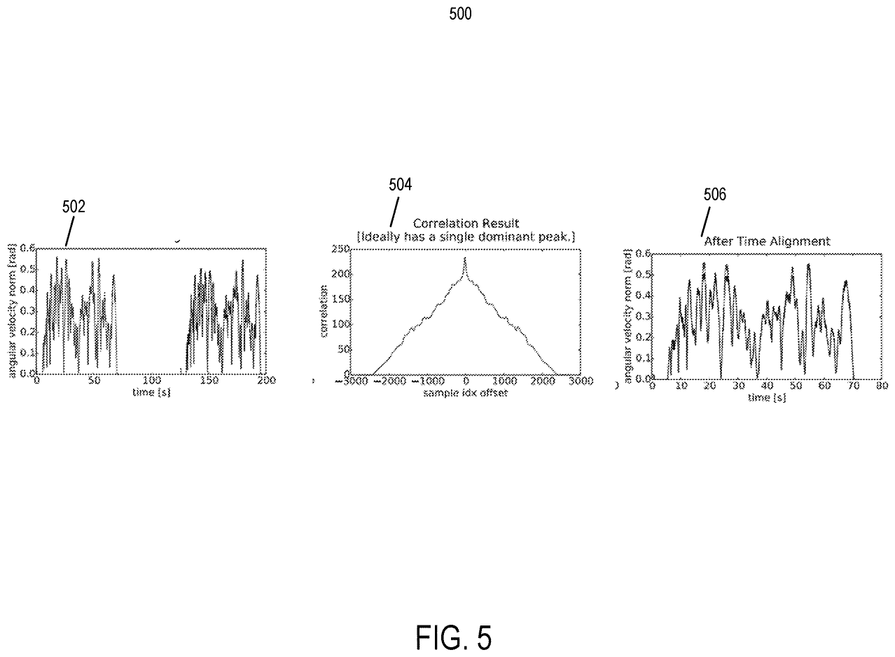

FIG. 5 depicts examples of plots 502, 504 and 506 showing the difference between temporally non-calibrated instrument and a temporally calibrated instrument, such as in a temporally synchronized stream. The plot 502 shows angular velocity over time before calibration or alignment, where two curves representing different data streams are offset from each other and not aligned. The two curves in plot 502 are showing time interval offset between each other. The plot 504 displays the correlation result, highlighting a single dominant peak that indicates the desired or optimal alignment point. At plot 506, upon recalibration, the angular velocity over time after alignment signals from the plot 502 are shown aligned and on top of each other, demonstrating successful recalibration. Plot 506 visually represents the effectiveness of the calibration process with respect to the uncalibrated plot 502, providing an example of improve synchronization in the data streams that can be used for accurate and reliable performance during medical procedures.

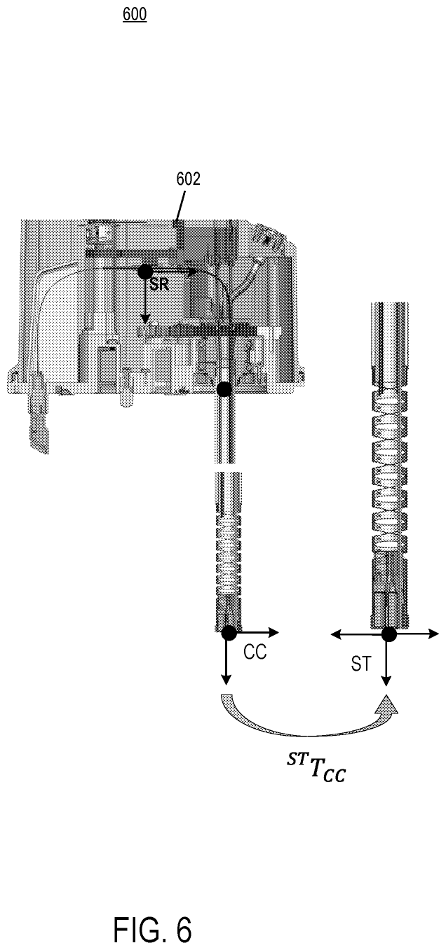

FIG. 6 illustrates an example of a configuration example 600 for a factory calibration. The configuration example can begin by moving the instrument 602 (e.g., endoscope) without actuating the robot joints. The configuration can then record images of a known calibration object (e.g., a checkerboard) using a camera while gathering shape sensor poses. The configuration can then perform time offset alignment between the shape sensor data and the camera images to provide synchronization. This can include correlating angular velocities between the two data streams to account for any temporal misalignment. The configuration can then use a hand-eye calibration method to estimate the transformation matrix (e.g., the compensation matrix) between the shape sensor tip and the camera which can be referred to as the ^{ST}T_{CC}. The ^{ref}T_{CC} can be represented as, or equal to an expression: ^{ref}T_{CB}*^{CB}T_{SR}*^{SR}T_{ST}*^{ST}T_{CC}, where, ST can represent the shape tip, SR can represent the shape sensor reference, ^{ref}T_{CC} can represent the final transformation matrix between the reference frame and the camera frame, ^{ref}T_{CB} can represent the transformation from the reference frame to the robot's base frame, ^{CB}T_{SR} can represent the transformation from the robot's base frame to the shape sensor reference frame, and ^{SR}T_{ST} can represent the transformation from the shape tip reference frame to the shape sensor reference frame. This combined transformation can provide the accurate calibration and alignment of the shape sensor and camera during the medical procedure.

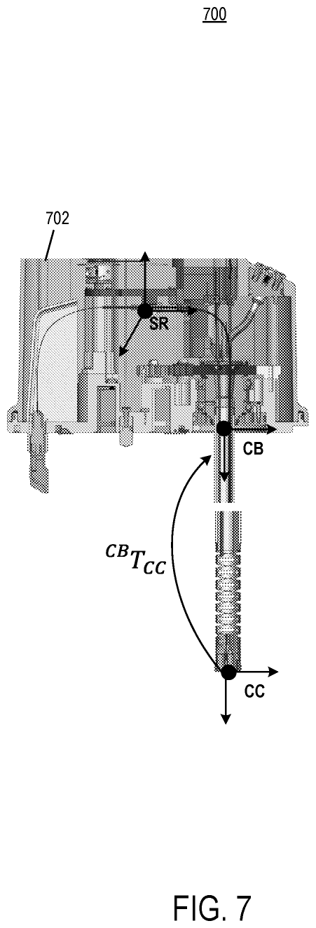

FIG. 7 illustrates an example of a configuration example 700 for performing robot to camera factory calibration. The configuration example 700 can begin by fixing the instrument 702 (e.g., an endoscope) in a static position (e.g., a jig) and move only the robot joints while capturing camera images of the calibration object. The configuration example 700 can then utilize kinematics data from the robot and extracted trajectory of the camera from the images to estimate the transformation between the catheter backend (CB) and the camera denoted as the ^{CB}T_{CC}. The time offset alignment can be utilized.

The configuration can then use a hand-eye calibration method to estimate the transformation matrix (e.g., the compensation matrix) between the shape sensor tip and the camera, referred to as the ^{ST}T_{CC}. The ^{ref}T_{CC} can be represented as ^{ref}T_{CB}*^{CB}T_{SR}*^{SR}T_{ST}*^{ST}T_{CC}, where ^{ref}T_{CC} can represent the final transformation matrix between the reference frame and the camera frame, CB can represent the catheter backend, ^{ref}T_{CB} can represent the transformation from the reference frame to the robot's catheter backend, ^{CB}T_{SR} can represent the transformation from the robot's base frame to the shape sensor reference frame, and ^{SR}T_{ST} can represent the transformation from the shape sensor reference frame to the shape sensor tip frame. This combined transformation can provide accurate calibration and alignment of the shape sensor and camera during the medical procedure.

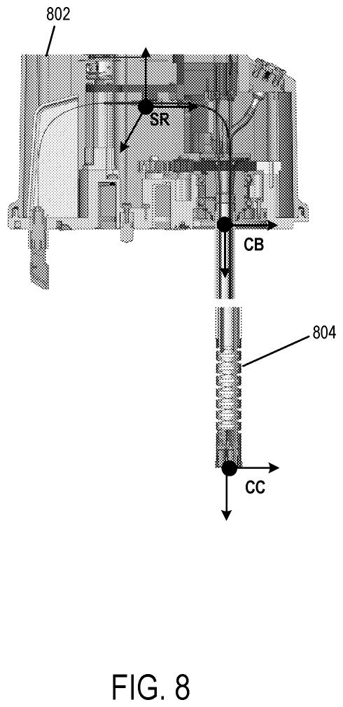

FIG. 8 illustrates an example of a configuration example 800 for performing final joint optimization for a factory calibration. The configuration example 800 can begin combining transformations by computing the matrix product for an instrument 802 using the following expression: ^{CB}T_{SR}=^{CB}T_{CC}*(^{SR}T_{CC})⁻¹ . The process can then include moving three components (robot, shape sensor and camera) and performing a non-linear optimization over the collected trajectories to further refine both spatial and temporal calibration parameters. This can ensure that the relationship between the camera, shape sensor, and robot kinematics in the multi-segment portion 804 of the instrument 802 is accurate to within a threshold value, reducing errors introduced by manufacturing variances or time delays.

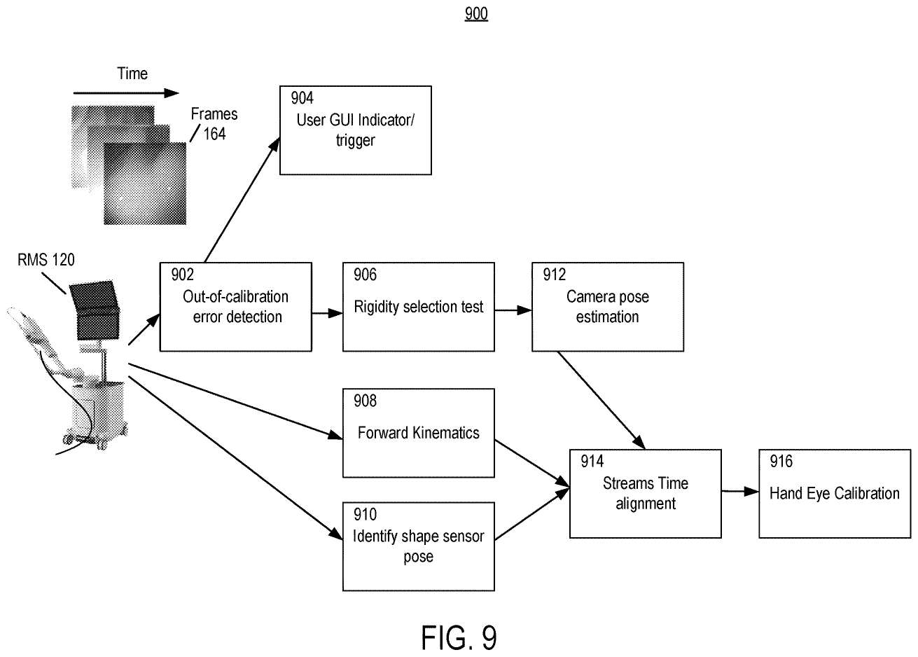

FIG. 9 illustrates an example flow diagram of a method 900 for providing an instrument recalibration during an ongoing medical procedure (e.g., an in-the-wild calibration). The method 900 can be implemented using, for example, example system 100 implemented using a computing system 300 of FIG. 3. The method 900 can include acts 902-916 implementing various operations of the spatiotemporal auto-recalibration of an instrument system 130, such as an endoscope used in a medical procedure.

At act 902, the method can begin with a robotic medical system detecting an out-of-calibration error. The out-of-calibration error can be generated in response to a comparison between instrument data (e.g., sensor and kinematics data for a multi-segment instrument) and a video data illustrating shape, movement and pose of the instrument. The out-of-calibration error can correspond to an error due to temporal mismatch between the instrument data and the video data. The out-of-calibration error can correspond to an error due to spatial mismatch between the instrument data and the video data. The comparison can be implemented using a deep learning module which can be configured to detect or identify the tool in the image. For example, during retroflection, the system can implement comparing the ruler markings over the endoscope to their expected projected positions using the kinematics and factory calibration information. The comparison can be utilized using an ML deep learning model. For instance, in response to the deep learning model determining that the markings do not coincide with the expected projected marking from the kinematics data, the system can determine, detect or identify that the system is out of calibration.

At 904, the method can provide a GUI indicator or a trigger indicating the out-of-calibration error. The GUI indicator or the trigger can include frames of a video data showing the instrument engaged in a medical procedure. This can involve displaying visual indicators or alerts to the medical team, informing them of the calibration status.

At act 906, the method can implement a rigidity selection test. This test can be performed to select or choose the frames for camera pose estimate at act 912. The method can select the frames that are valid (e.g., filter the frames to select clear or crisp frames) which then can be used for both estimating the camera position and also reconstructing a 3D representation of the environment. The rigidity selection test can help in identifying the appropriate calibration parameters for the instrument system.

At act 908, the method can include forwarding kinematics data from the robotic medical system (RMS) 120 to the data processing system 150. The kinematics data can be used to determine the expected position and orientation of the instrument based on the movement data of its segments. This data can be used in predicting the endoscope's pose and aligning it with the actual sensor data.

At act 910, the method can include identifying the shape sensor pose. This can involve capturing and processing data from the shape sensors attached to the instrument system (e.g., endoscope) to determine its actual pose. The shape sensor pose data can then be compared with the forward kinematics data to detect any discrepancies.

At act 912, the method can implement camera pose estimation. This can involve determining or estimating the camera tip position from the video. The camera pose estimation can help in aligning the video data with the pose information from the shape sensor system.

At act 914, the method can include streams time alignment. This can involve synchronizing the timestamps of the video data and the sensor data to ensure accurate alignment. This step ensures that the data from different sources is accurately aligned in time, allowing for improved calibration.

At act 916, the method can perform hand-eye calibration. This can involve aligning the endoscope's pose with the camera's view to ensure that the visual data accurately reflects the endoscope's movements. This can be done using a factory matrix, compensation matrix or the combination of both to provide values or settings for synchronization of the instrument data and the video data. The hand-eye calibration can allow for spatial and temporal alignment in a calibrated manner.

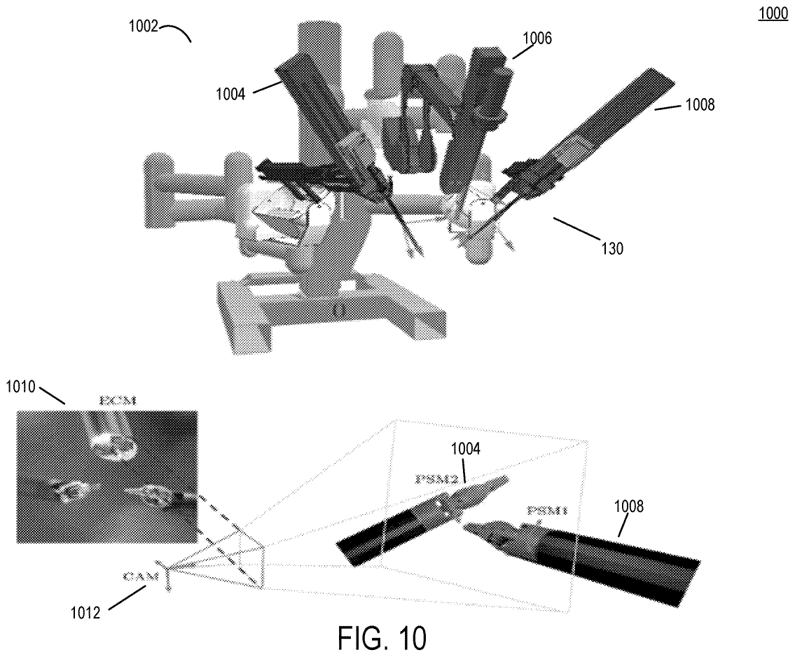

FIG. 10 illustrates an example 1000 of a recalibrated instrument illustrated from viewpoints 1002 and 1010. At 1002, the instrument can provide multiple handling devices, such as 1004, 1006 and 1008 handling different medical instruments 112 during a procedure. The handling devices can include handling or manipulator arms 235 of a robotic medical system 120. At view 1010, an image capture device (e.g., a camera) can provide a viewpoint 1012 for viewing the handling devices 1004 and 1008 within an image or video frame 164.

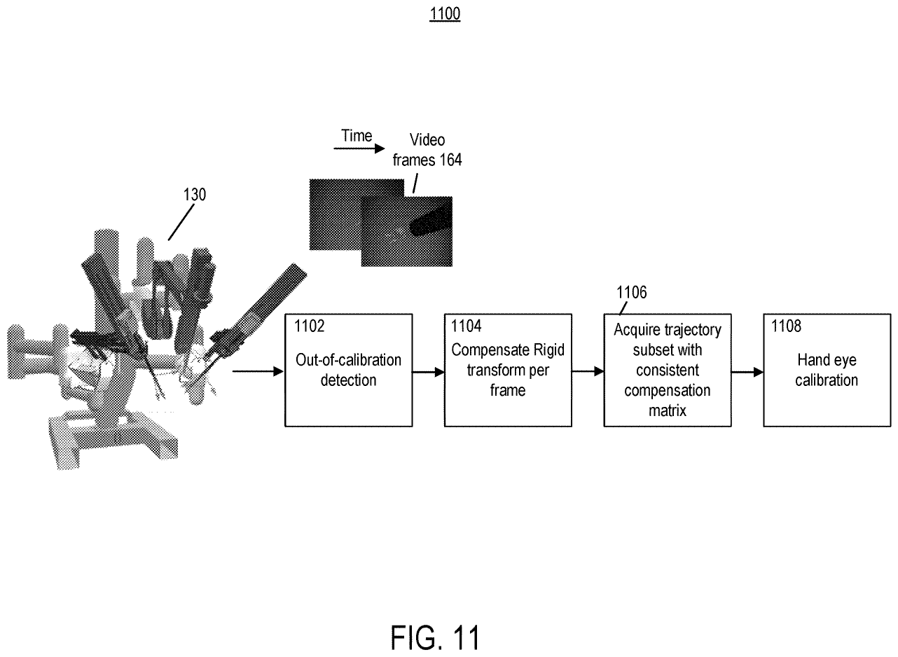

FIG. 11 illustrates an example 1100 of a flow diagram of a recalibration of an instrument during a procedure. The method 1100 can be implemented using, for example, example system 100 implemented using a computing system 300 of FIG. 3. The method 1100 can include acts 1102-1112 implementing various operations of the spatiotemporal autocalibration of an instrument system 130, such as an endoscope used in a medical procedure.

At act 1102, the method can begin with a system 100 detecting an out-of-calibration error from the instrument. This can involve identifying discrepancies between the pose information from the kinematics data and the video data from the cameras. For instance, the method can be applied to any robotic system, including for example a flexible endoscope or a system with end effector with medical instruments attached. The detection can be based on comparing the data streams and identifying any misalignment.

At act 1104, the method can include compensating for the detected out-of-calibration error by applying a rigid transform per frame. For instance, the method can include generating and applying a compensation matrix to align the pose information with the video data. This compensation can be performed in real-time to ensure accurate calibration during the procedure.

At act 1106, the method can include acquiring a trajectory subset with consistent compensation matrices. The method can include using this subset to refine the calibration process. For example, the trajectory subset can include data points that are used to adjust the alignment of the pose information and video data.

At act 1108, the method can perform hand-eye calibration. The system can use a hand-eye calibration method to estimate the transformation matrix (e.g., the compensation matrix) between the shape sensor tip and the camera. A combined transformation can be implemented to account for a plurality of segments of an instrument and provide accurate calibration and alignment of the shape sensor and camera during the medical procedure.

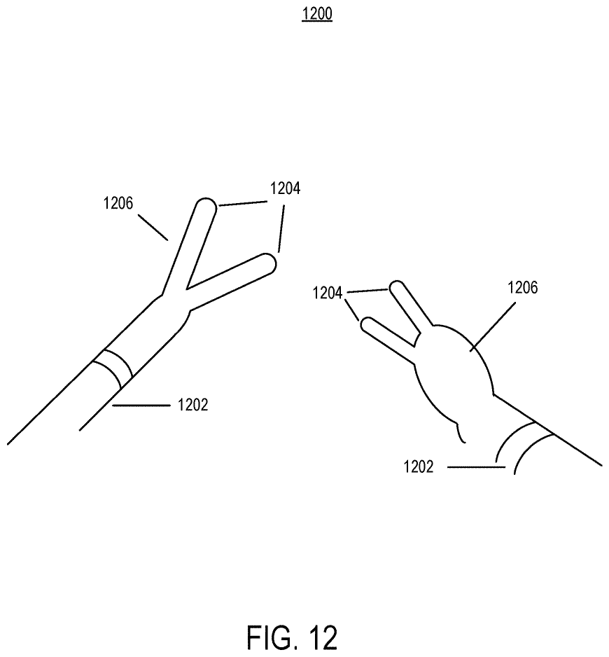

FIG. 12 illustrates an example 1200 of an instrument system configured for grabbing or holding patient tissues (e.g., forceps configured for a robotic medical system) that can be calibrated using the techniques described herein. In example 1200, a pair of forceps instruments 1202 can be adapted for control and use by a robotic medical system and can be calibrated or recalibrated during use. Each of the forceps 1202 can include a pair of tips 1204 to form a jaw 1206 for grabbing and holding tissues during a procedure. The technical solutions of the present disclosure can be utilized to recalibrate the positioning of each of the tips 1204 in order to adjust (e.g., temporally or spatially) the positioning of the tips 1204 to allow for improved handling of the instruments.

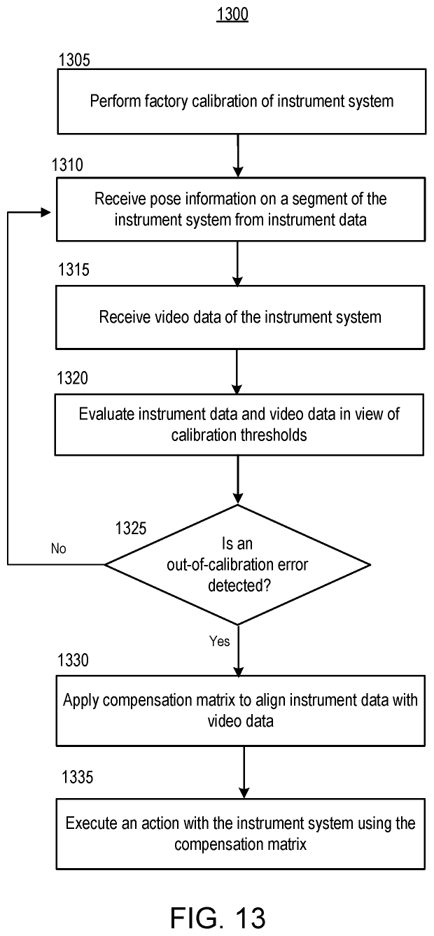

FIG. 13 illustrates an example flow diagram of a method 1300 for providing any combination of a spatial or temporal autocalibration of a multi-segment instrument system for a medical procedure. The method 1300, can be performed by a system having one or more processors (e.g., 310) configured to perform acts or operations of the system 100 by executing computer-readable instructions stored on a memory (e.g., 315). For instance, method 1300 can be implemented using a non-transitory computer readable medium storing instructions that, when executed by one or more processors (e.g., 310), cause the one or more processors (e.g., 310) to implement operations or acts of the method. The method 1300 can be performed, for example, in accordance with any features or techniques discussed in connection with FIGS. 1-3.

The method 1300 can include operations 1305-1330. At act 1305, the method can perform a factory calibration of an instrument system. At act 1310, the method can receive pose information on a segment of the instrument system from the instrument data. At 1315, the method can receive video data of the instrument system. At 1320, the method can evaluate the instrument data and the video data in view of the calibration thresholds. At 1325, the method can determine if an out-of-calibration error is detected. At 1330, the method can apply a compensation matrix to align the instrument data with the video data. At 1335, the method can execute an action with the instrument system using the compensation matrix.

At act 1305, the method can perform a factory calibration of an instrument system. The one or more processors executing a data processing system can generate and apply a factory calibration to the instrument system to maintain the video data and the instrument data within calibration thresholds (e.g., in a calibrated state). The factory calibration can utilize a factory matrix that can be generated to align temporally or spatially the video data and the instrument data of the instrument attached to the robotic medical system and used during the medical procedure. The factory matrix can include an array of values indicating a transformation for aligning, comparing or matching the video data (e.g., video frames) with the sensor data or kinematics data of the multi-segment instrument system utilized during the robotic medical procedure.

The instrument system can include a shape sensor comprising one or more sensors or detectors for monitoring positioning (e.g., pose), movement, velocity, acceleration or state of the instrument system (e.g., multi-segment endoscopic device). The instrument system can include or be coupled with the vision system. The shape sensor and the video system can be calibrated prior to performance of the medical procedure using a factory-calibrated transform matrix. The factory-calibrated transform matrix can be different from the compensation matrix used during the medical procedure responsive to detection of the out-of-calibration error. For instance, the factory matrix can include a set of entries arranged in one or more rows and columns of values that are different than the values for the same set of entries in the same number of rows and columns of the compensation matrix. For instance, the compensation matrix can include a different number of rows and columns (e.g., a different set of entries) than the factory matrix.

At act 1310, the method can receive pose information on a segment of the instrument system from the instrument data. The method can include the one or more processors receiving, from a shape sensor system, pose information related to a tip of one or more segments along a flexible body of an instrument system used to perform a medical procedure. For instance, the one or more processors can receive instrument data (e.g., sensor or kinematics data) indicative of the positioning, state, location, orientation, pose or movement of one or more segments of the instrument system. For instance, the method can include receiving, from one or more sensors, kinematics information during the medical procedure information from which the calibrator can determine the pose information (e.g., the state or orientation or pose of the plurality of segments of the instrument).

At 1315, the method can receive video data of the instrument system. The method can include the one or more processors receiving, from a vision system, a video stream related to the medical procedure. The one or more processors can receive, for example, from a camera of the instrument system (e.g., endoscopic device) one or more frames of a video stream. The one or more processors can receive one or more frames of a video stream of an external data capture device monitoring the body of the patient and the movement of the instrument within the body. The one or more processors can receive the one or more frames of a data capture device that includes an X-ray imaging device, a magnetic resonance imaging device, an ultrasound device, a depth imaging device, a computed tomography scanner or a positron emission tomography scanner. The video data received can include any series of frames that include an image of at least a portion of the instrument device and that include metadata including timestamps indicating the time at which the image frames were captured.

At 1320, the method can evaluate the instrument data and the video data in view of the calibration thresholds. The method can include the one or more processors determining an amount of difference or discrepancy between the positioning of a portion of the instrument device (e.g., a tip of a segment of the instrument device) according to instrument data (e.g., sensor or kinematics data) and the position of the same portion of the instrument device according to the video data (e.g., image frame location). The positioning can be determined with respect to locations determined according to the instrument data and the video data, or with respect to a reference point. This amount of difference can then be compared with a calibration threshold to determine if the system is out of spatial calibration.

The method can include the one or more processors determining an amount of difference or discrepancy between the timing of a portion of the instrument device (e.g., a tip of a segment of the instrument device) according to instrument data (e.g., sensor or kinematics data) and the timing of the same portion of the instrument device according to the video data (e.g., image frame location). The timing can be with respect to a location or position being reached or achieved. This amount of difference in timing can then be compared with a calibration threshold for timing calibration to determine if the system is out of timing calibration. The method can include identifying a type of task performed during the medical procedure. The method can include selecting a calibration threshold based on the type of task. The method can include determining the out-of-calibration error based on the calibration threshold selected for the type of task. The out-of-calibration error can be determined in response to the calibration threshold being exceeded (e.g., by the spatial difference or timing difference between the two data streams exceeding the given calibration (e.g., spatial or temporal) threshold.

At 1325, the method can determine if an out-of-calibration error is detected. The one or more processors can include detecting, during the medical procedure, an out-of-calibration error between one or more frames of the video stream and the pose information. The calibrator of the data processing system can determine the out-of-calibration error (e.g., for spatial or temporal calibration) based on the spatial or temporal calibration threshold being satisfied or exceeded at act 1320. For instance, a calibrator can determine the presence or occurrence of a positioning out-of-calibration error in response to a difference or mismatch in the positioning of a portion (e.g., a tip) of a particular segment of a multi-segment instrument system determined based on the instrument data and the video data exceeding the positioning calibration threshold. When such a difference or mismatch exceeds the positioning calibration threshold, the error detector can determine a positioning out-of-calibration error to initiate a recalibration.

For instance, a calibrator can determine the presence or occurrence of a timing or temporal out-of-calibration error in response to a difference or mismatch in the timing of a portion (e.g., a tip) of a particular segment of a multi-segment instrument system determined based on the instrument data and the video data exceeding the temporal calibration threshold. When such a difference or mismatch exceeds the temporal calibration threshold, the error detector can determine a temporal out-of-calibration error to initiate a recalibration. The temporal calibration can be implemented using or based at least on the timestamps of the video frames or the timestamps of the instrument data, or both. For instance, a compensation matrix can be generated to offset or adjust the timing of the instrument data and the video data such that they are within the calibration thresholds.

For instance, the method can include receiving, from one or more sensors, kinematics information during the medical procedure. The method can include detecting an out-of-calibration error between the kinematics information and at least one of the pose information or the video stream. The method can include detecting the out-of-calibration error based on camera intrinsic parameters.

The method can include generating a compensation matrix in response to the out-of-calibration error (e.g., whether spatial, temporal or spatial and temporal together). The compensation matrix can include values or entries to reset or adjust (e.g., temporally, spatially or both) the instrument data with the video data such that their differences are within (e.g., satisfy) the calibration thresholds. The compensation matrix can be utilized instead of the factory matrix to recalibrate the instrument and the video data. The compensation matrices can be utilized together with (e.g., in conjunction with) the factory matrix or matrices in order to bring the instrument data and the video data within calibration thresholds or tolerance.

At 1330, the method can apply a compensation matrix to align the instrument data with the video data. The method can include applying, responsive to detection of the out-of-calibration error during the medical procedure, a compensation matrix to at least one of the pose information or the video stream to align the pose information with the video stream. The method can include applying the compensation matrix to align timestamps associated with frames of the pose information and frames of the video stream.

The method can generate and apply multiple compensation matrices for multiple calibrations, such as temporal, spatial or a combination of spatial and temporal. The compensation matrices can be generated based on the types of data (e.g., a first compensation matrix for a sensor data and a second compensation matrix for a kinematics data or a video data). The compensation matrices can be generated based on the type of instrument utilized (e.g., a particular type of an endoscope or a particular tool utilized). For instance, the method can include applying a second compensation matrix to the kinematics information to align frames of the kinematics information with frames of the at least one of the pose information or the video stream. For instance, the method can include applying the second compensation matrix to align timestamps of the frames of the kinematics information with timestamps of the frames of the at least one of the pose information or the video stream. For instance, the method can include applying the second compensation matrix to perform hand eye calibration of the frames of the kinematics information with the frames of the at least one of the pose information or the video stream.

The one or more processors can select a calibration technique to apply based on the type of task and determine the compensation matrix to apply based on the selected calibration technique. For instance, the calibration technique can include a rigid transform per frame. For instance, the calibration technique can include an interactive parametric model configured based on a state of a tool coupled with the instrument system.

At 1335, the method can execute an action with the instrument system using the compensation matrix. The one or more processors can execute any type of actions utilizing the compensation matrix, either instead of the factory matrix or together with the factory matrix, to perform actions of the medical procedure while having the instrument data and the video data perform within the calibration thresholds. For instance, the data processing system can execute an action related to the medical procedure using the pose information aligned with the video stream based on the compensation matrix. One or more instrument controllers can perform movements and operations using the instruments or tools in accordance with the one or more compensation matrices.

The robotic medical system can implement various actions based on the recalibration. For example, the method can include activating, subsequent to application of the compensation matrix, a digital ruler to measure a dimension of an anatomical structure related to the medical procedure. The method can include providing, for display via a display device, an indication of a measurement of the dimension made using the digital ruler. The method can include registering, based on calibrated pose information and the video stream using the compensation matrix, a virtual object related to the medical procedure. The method can include displaying, based on calibrated pose information and the video stream using the compensation matrix, a virtual annotation with the video stream of the medical procedure.

In some cases, the data processing system can determine to block or pause the performance of an action related to movement of the instrument based on, or responsive to, detecting an out-of-calibration error. For example, the data processing system can determine to pause the performance of an action while the pose information and video data stream are being calibrated based on application of the compensation matrix.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

With respect to the use of plural or singular terms herein, those having skill in the art can translate from the plural to the singular or from the singular to the plural as is appropriate to the context or application. The various singular/plural permutations can be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description can illustrate a specific order of method steps, the order of such steps can differ from what is depicted and described, unless specified differently above. Also, two or more steps can be performed concurrently or with partial concurrence, unless specified differently above. Such variation can depend, for example, on the software and hardware systems chosen and on designer choice. All such variations are within the scope of the disclosure. Likewise, software implementations of the described methods can be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or can be acquired from practice of the disclosed implementations. It is intended that the scope be defined by the claims appended hereto and their equivalents.

What is claimed is:

1. A system, comprising:
one or more processors, coupled with memory, to:
receive, from a shape sensor system, pose information related to a tip of one or more segments along a flexible body of an instrument system used to perform a medical procedure;
receive, from a vision system, a video stream related to the medical procedure;
detect, during the medical procedure, an out-of-calibration error between one or more frames of the video stream and the pose information;
apply, responsive to detection of the out-of-calibration error during the medical procedure, a compensation matrix to at least one of the pose information or the video stream to align the pose information with the video stream; and
execute an action related to the medical procedure using the pose information aligned with the video stream based on the compensation matrix.

2. The system of claim 1, wherein the shape sensor and the vision system are calibrated prior to performance of the medical procedure using a factory-calibrated transform matrix, wherein the factory-calibrated transform matrix is different from the compensation matrix used during the medical procedure responsive to detection of the out-of-calibration error.

3. The system of claim 1, wherein the one or more processors are further configured to:
temporally synchronize frames of the pose information and frames of the video stream by applying one or more offsets to one or more timestamps associated with at least one of the frames of the pose information or the frames of the video stream.

4. The system of claim 1, wherein the one or more processors are further configured to:
receive, from one or more sensors, kinematics information during the medical procedure;
detect an out-of-calibration error between the kinematics information and at least one of the pose information or the video stream; and
apply a second compensation matrix to kinematics data to align the kinematics data with frames of the at least one of the pose information or the video stream.

5. The system of claim 4, wherein the one or more processors are further configured to:
apply the second compensation matrix to sensor data to align the sensor data with the frames of the at least one of the pose information or the video stream.

6. The system of claim 4, wherein the one or more processors are further configured to:
apply the second compensation matrix to perform hand eye calibration of the frames of the kinematics information with the frames of the at least one of the pose information or the video stream.

7. The system of claim 1, wherein the one or more processors are further configured to:
activate, subsequent to application of the compensation matrix, a digital ruler to measure a dimension of an anatomical structure related to the medical procedure; and
provide, for display via a display device, an indication of a measurement of the dimension made using the digital ruler.

8. The system of claim 1, wherein the one or more processors are further configured to:
register, based on calibrated pose information and the video stream using the compensation matrix, a virtual object related to the medical procedure.

9. The system of claim 1, wherein the one or more processors are further configured to:
display, based on calibrated pose information and the video stream using the compensation matrix, a virtual annotation with the video stream of the medical procedure.

10. The system of claim 1, wherein the one or more processors are further configured to:
detect the out-of-calibration error based on camera intrinsic parameters.

11. The system of claim 1, wherein the one or more processors are further configured to:
identify a type of task performed during the medical procedure;
select a calibration threshold based on the type of task; and
determine the out-of-calibration error based on the calibration threshold selected for the type of task.

12. The system of claim 11, wherein the one or more processors are further configured to:
select a calibration technique to apply based on the type of task; and
determine the compensation matrix to apply based on the selected calibration technique.

13. The system of claim 12, wherein the calibration technique comprises a rigid transform per frame.

14. The system of claim 12, wherein the calibration technique comprises an interactive parametric model configured based on a state of a tool coupled with the instrument system.

15. A method, comprising:
receiving, by one or more processors coupled with memory, from a shape sensor system, pose information related to a tip of one or more segments along a flexible body of an instrument system used to perform a medical procedure;
receiving, by the one or more processors, from a camera, a video stream related to the medical procedure;
detecting, by the one or more processors, during the medical procedure, an out-of-calibration error between one or more frames of the video stream and the pose information;
calibrating, by the one or more processors, responsive to detection of the out-of-calibration error during the medical procedure, the pose information with the video stream using a compensation matrix; and
providing, by the one or more processors, for display via a display device, a location of the tip aligned based on application of the compensation matrix.

16. The method of claim 15, comprising:
temporally synchronizing, by the one or more processors, frames of the pose information and frames of the video stream by applying one or more offsets to one or more timestamps associated with at least one of the frames of the pose information or the frames of the video stream.

17. The method of claim 15, comprising;
receiving, by the one or more processors, from one or more sensors, kinematics information during the medical procedure;
detecting, by the one or more processors, an out-of-calibration error between the kinematics information and at least one of the pose information or the video stream; and
applying, by the one or more processors, a second compensation matrix to the kinematics information to align frames of the kinematics information with frames of the at least one of the pose information or the video stream.

18. The method of claim 15, comprising:
activating, by the one or more processors, subsequent to application of the compensation matrix, a digital ruler to measure a dimension of an anatomical structure related to the medical procedure; and
providing, for display via the display device, an indication of a measurement of the dimension made using the digital ruler.

19. The method of claim 15, comprising:
registering, by the one or more processors, based on the calibrated pose information and the video stream using the compensation matrix, a virtual object related to the medical procedure.

20. A non-transitory computer-readable medium storing processor-executable instructions that, when executed by one or more processors, cause the one or more processors to:
receive, from a shape sensor system, position and orientation information related to a tip of one or more segments along a flexible body of an instrument system used to perform a medical procedure;
receive, from a sensor, a video stream related to the medical procedure;
detect, during the medical procedure, an out-of-calibration error between one or more frames of the video stream and the position and orientation information;
align, responsive to detection of the out-of-calibration error during the medical procedure, the position and orientation information with the video stream using a compensation matrix; and
perform an action related to the medical procedure using the aligned position and orientation information with the video stream.
